(19) United States
(12) Patent Application Publication
(10) Pub. No.: US 2009/0215873 A1
(43) Pub. Date: Aug. 27, 2009

(54) Therapeutic Molecules and their Uses

(76) Inventors: Andrew Vaillant, Roxboro (CA); Jean-Marc Juteau, Blainville (CA)

Correspondence Address: OGILVY RENAULT LLP, 1, Place Ville Marie, SUITE 2500, MONTREAL, QC, H3B 1R1, CA

(21) Appl. No.: 11/992,891

(22) PCT Filed: Jul. 18, 2006
(86) PCT No.: PCT/CA2006/001177

§ 371 Date: Apr. 23, 2009

Related U.S. Application Data
(60) Provisional application No. 60/721,570, filed on Sep. 29, 2005.

(51) Int. Cl. A61K 31/7088

(52) U.S. Cl. 514/44 R

(57) ABSTRACT

The present invention relates with the identification and use of oligonucleotides acting by a sequence independent mode of action for the prevention and treatment of thrombotic disorders, cholesterol related disorders, dyslipidemia, osteoporosis and snake venom effects.

### FIELD OF THE INVENTION

The present invention relates with the identification and use of oligonucleotides acting by a sequence independent mode of action for the prevention and treatment of thrombotic disorders, cholesterol related disorders, dyslipidemia, osteoporosis and snake venom effects.

### BACKGROUND OF THE INVENTION

Thrombotic disorders, cholesterol and dyslipidemia related disorders, osteoporosis and snake venom effects have in common that they are blood related diseases, disorders or conditions, or have blood related constituents. Indeed, thrombotic disorders are by definition blood related conditions disorders involving mainly the coagulation or blood clotting. In the case of cholesterol related disorders and dyslipidemia, important constituents involved in these disorders are blood related. In fact, lipids, lipoproteins, apolipoproteins, lipid complex (LDL, VLDL), cholesterol, free fatty acids, triglycerides and cytokines are found in the blood. Concerning osteoporosis, some constituents of the disease are blood related as for the example of serum testosterone, serum estradiol, serum parathyroid hormone, serum lactoferrine, serum vitamin D, serum calcium, and serum phosphate. In the case of snake venom effects, this condition has important blood related constituents. For example, snake venoms can be classified as hemotoxic (attacking tissue and blood) and envenoming bites may result in coagulopathies, hemorrhage, hemolysis, hypofibrinogenemia and thrombocytopenia.

### Thrombotic Disorders

The coagulation or blood clotting process is involved both in normal hemostasis where the clot stops blood loss from a damaged blood vessel and in abnormal thrombosis where the clot blocks circulation through a blood vessel. During normal hemostasis, the platelets adhere to the injured blood vessel and aggregate to form the primary hemostatic plug. The platelets then stimulate local activation of plasma coagulation factors, leading to generation of a fibrin clot that reinforces the platelet aggregate. Later, as wound healing occurs, the platelet aggregate and fibrin clot are degraded by specifically activated proteinases. During the pathological process of thrombosis, the same mechanisms create a platelet/fibrin clot that occludes a blood vessel. Arterial thrombosis may produce ischemic necrosis of the tissue supplied by the artery, e.g., myocardial infarction due to thrombosis of a coronary artery, or stroke due to thrombosis of a cerebral artery. Venous thrombosis may cause the tissues drained by the vein to become edematous and inflamed, and thrombosis of a deep vein may result in a pulmonary embolism.

The endpoint of the coagulation process is the generation of a powerful serine protease, thrombin, which cleaves the soluble plasma protein fibrinogen so that an insoluble meshwork of fibrin strands develops, enmeshing red cells and platelets to form a stable clot. This coagulation process can be triggered by injury to the-blood vessels and involves the rapid, highly controlled interaction of more than 20 different coagulation factors and other proteins to amplify the initial activation of a few molecules to an appropriately sized, fully developed clot.

Four main types of therapies are used to prevent or treat thrombosis: antiplatelet agents, anticoagulant agents (e.g. heparin), vitamin K antagonists (e.g. coumarin derivatives) and thrombolytic agents. Each type of agent interferes with clotting at a different step in the coagulation pathway. In general, antiplatelet agents are used in prophylaxis against arterial thrombosis, because platelets are more important in initiating arterial than venous thrombi. Bleeding is the primary adverse effect of heparin. Major bleeding occurs in 1% to 33% of patients who receive various forms of heparin therapy. Purpura, ecchymoses, hematomas, gastrointestinal hemorrhage, hematuria, and retroperitoneal bleeding are regularly encountered complications of heparin therapy. Bleeding is the major adverse effect of vitamin K antagonists. Especially serious episodes involve irreversible damage resulting from compression of vital structures or from massive internal blood loss.

There exists a need in the art for compounds, methods of treatment and formulations capable of exerting anticoagulant or thrombolytic effects without severe adverse side effects, and methods and compositions capable of improving the therapeutic effectiveness of existing anticoagulant or thrombolytic agents, which ideally could reduce the required dosages of such existing agents

### Cholesterol and Dyslipidemia Related Disorders

The evidence linking elevated serum cholesterol to coronary heart disease is overwhelming. Circulating cholesterol is carried by plasma lipoproteins, which are particles of complex lipid and protein composition that transport lipids in the blood. Low density lipoprotein (LDL) and high density lipoprotein (HDL) are the major cholesterol-carrier proteins. LDL is believed to be responsible for the delivery of cholesterol from the liver, where it is synthesized or obtained from dietary sources, to extrahepatic tissues in the body. The term “reverse cholesterol transport” describes the transport of cholesterol from extrahepatic tissues to the liver, where it is catabolized and eliminated. It is believed that plasma HDL particles play a major role in the reverse transport process, acting as scavengers of tissue cholesterol. HDL is also responsible for the removal of non-cholesterol lipid, oxidized cholesterol and other oxidized products from the bloodstream.

Atherosclerosis, for example, is a slowly progressive disease characterized by the accumulation of cholesterol within the arterial wall. Compelling evidence supports the belief that lipids deposited in atherosclerotic lesions are derived primarily from plasma apolipoprotein B (apo B)-containing lipoproteins, which include chylomicrons, CLDL, intermediate-density lipoproteins (IDL), and LDL. The apo B-containing lipoprotein, and in particular LDL, has popularly become known as the “bad” cholesterol. In contrast, HDL serum levels correlate inversely with coronary heart disease. Indeed, high serum levels of HDL are regarded as a negative risk factor. It is hypothesized that high levels of plasma HDL are not only protective against coronary artery disease, but may actually induce regression of atherosclerotic plaques. Thus, HDL has popularly become known as the “good” cholesterol. Apolipoproteins involved in cholesterol activity include apolipoprotein B.

A common gene affecting LDL cholesterol levels is ApoE, which has three major variations, E2, E3, and E4. The most common form, E3, occurs in approximately 78% of the population, while E4 has a frequency of 15%, and E2 a frequency of 7%. Studies have established that, compared to the E3 allele, E4 is associated with higher levels of LDL cholesterol and apo B; and E2 is associated with lower levels of LDL cholesterol and apo B.

In the past two decades, the segregation of cholesterolemic compounds into HDL and LDL regulators and recognition of the desirability of decreasing blood levels of the latter has led to the development of a number of drugs. However, many of these drugs have undesirable side effects and/or are contraindicated in certain patients, particularly when administered in combination with other drugs. Therefore, there is a need for compounds, methods of treatment and formulations for cholesterol related disease control.

The metabolic syndrome (METS) or insulin resistance/metabolic syndrome are characterized by the variable coexistence of hyperinsulinaemia, obesity, dyslipidemia, and hypertension. Dyslipidemia, the hallmark of the METS, is summarized as (a) increased flux of free fatty acids, (b) raised triglycerides values, (c) low high density lipoprotein (HDL) cholesterol values, (d) increased small, dense low density lipoprotein (LDL) values, and (e) raised apolipoprotein (apo) B values. Dyslipidemia is widely established as an independent risk factor for cardiovascular disease.

Resistin, tumour necrosis factor-alpha, interleukin 6 (IL-6) and interleukin 1 (IL-1) are believed to be involved in metabolic disorders such as the metabolic syndrome and obesity.

Overweight or obesity increases the risk of many diseases and health conditions, including hypertension, dyslipidemia (for example, high total cholesterol or high levels of triglycerides), type 2 diabetes and CHD. Dyslipidemia can be defined as disorders in the lipoprotein metabolism including hypercholesterolemia, liypertriglyceridemia, combined hyperlipidemia, and low levels of high-density lipoprotein (HDL) cholesterol. All of the dyslipidemias can be primary or secondary. Both elevated levels of low-density lipoprotein (LDL) cholesterol and low levels of HDL cholesterol predispose to premature atherosclerosis.

Adipose tissue, for a long time, was regarded as a comparatively passive side of energy storage (accumulated in the form of triglycerides). However, studies show that adipose tissue is an endocrine organ producing various proteins (adipocytokines). Adipocytokines include leptin, angiotensinogen, tumour necrosis factor, interleukin 6, plasminogen activator-inhibitor 1, transforming growth factor beta, adipsin, adiponectin and resistin.

Observational studies in patients with coronary heart disease (CHD) have consistently shown a strong association of increased triglyceride with CHD. Patients with diabetes, central obesity, peripheral vascular disease, hypertension, and chronic renal disease, which are known to be associated with an increased risk of CHD, should have triglyceride levels measured.

### Osteoporosis

Osteoporosis is a disease that is characterized by a decrease in bone mass and density, causing bones to become fragile. Bone is a metabolically active and highly organized connective tissue. The main functions of bone is the provision of mechanical and structural support, maintaining blood calcium levels, supporting haematopoiesis and housing the important vital organs such as brain, spinal cord and heart. To accomplish these functions bone needs continuous remodeling. Bone contains two distinct cell types, osteoblasts which are essential for bone formation (synthesis) and osteoclasts which are essential for bone resorption (break down). Morphogenesis and remodeling of bone involves the synthesis of bone matrix by osteoblasts and coordinated resorption by osteoclasts. Syncytia formed after the fusion of mononuclear phagocytes are called osteoclasts. Multinucleation via cell fusion appears to endow monocytes/macrophages with the capacity to digest and resorb extracellular infectious agents, foreign materials, and other components that are too large to be internalized. The presence of osteoclasts is a hallmark of granulomas, which are formed in inflammatory sites of tuberculosis, fungal infection, HIV infection, sarcoidosis, Crohn's disease, and tumors. The physiological role of osteoclasts still remains unknown, but possible roles in the host defense against bacterial infection have been suggested. Osteoclasts are formed by the fusion of mononuclear progenitors of the monocyte/macrophage lineage. These polykaryons are characterized by the presence of tartrate-resistant acid phosphatase activity and have a crucial role not only in physiological bone remodeling, but also in local bone disorders such as osteoporosis and bone tumors.

The current known therapeutic agents for osteoporosis have a variety of associated disadvantages. The side effects of current therapies include an elevated risk of breast and uterine cancers, upper gastrointestinal distress and induction of immune responses. Therefore, there is a need for compounds, methods of treatment and formulations for prevention and treatment of osteoporosis

### Snake Venom Effects

7000 venomous snake bites are reported annually in the United States. Poisonous snake bites include bites by any of the following: rattlesnake, copperhead, cottonmouth (water moccasin) and coral snake. Antivenins can be used for treatment but they are usually specific to venom from a single snake species requiring stockpiling of multiple antivenins in endemic areas. Antivenins may also have important side-effects. There is a need for compounds, methods of treatment and formulations for prophylaxis or treatment of snake venom effect in humans and animals. The availability of a universal drug that can treat different snake bites would be useful.

It would be useful to have sequence independent ONs, formulations and methods of treatment incorporating such ONs for the prevention and treatment of thrombotic disorders, cholesterol related disorders, dyslipidemia, osteoporosis and snake venom effects.

### SUMMARY OF THE INVENTION

The invention relates to therapeutic oligonucleotides (ONs) acting predominantly by a sequence independent mode of action. The invention also relates to ONs and their use as therapeutic agents, and more particularly for their use in methods of treatment and formulations for the treatment diseases.

In accordance with the present invention, there is provided a therapeutic oligonucleotide formulation comprising at least one oligonucleotide having a therapeutic activity against a target disease, said activity occurring principally by a sequence independent mode of action.

In an additional embodiment, the oligonucleotide formulation of the present invention comprises an oligonucleotide of at least 15 nucleotides in length; 20 nucleotides in length; 25 nucleotides in length; 30 nucleotides in length; 35 nucleotides in length; preferably 40 nucleotides in length; 45 nucleotides in length; 50 nucleotides in length; more preferably 60 nucleotides in length; or 80 nucleotides in length.

In a further embodiment, the oligonucleotide formulation comprises an oligonucleotide of 20-30 nucleotides in length; 30-40 nucleotides in length; preferably 40-50 nucleotides in length; 50-60 nucleotides in length; more preferably 60-70 nucleotides in length; or 70-80 nucleotides in length.

In a further embodiment, the oligonucleotide formulation of the present invention comprises an oligonucleotide which is not complementary to any equal length portion of a microbial genomic sequence. More preferably, the genomic sequence is of a human or of a non human animal.

In accordance with the present invention, there is provided an oligonucleotide formulation comprising an oligonucleotide containing at least 10 contiguous nucleotides of randomer sequence; more preferably 20 nucleotides of randomer sequence; 30 nucleotides of randomer sequence; or 40 nucleotides of randomer sequence.

In a further embodiment, the oligonucleotide formulation of the present invention comprises a randomer oligonucleotide.

In another embodiment of the present invention, the oligonucleotide formulation comprises an oligonucleotide having a homopolymer sequence of at least 10 contiguous A nucleotides; 10 contiguous T nucleotides; 10 contiguous U nucleotides; 10 contiguous G nucleotides; 10 contiguous I nucleotide analogs; or 10 contiguous C nucleotides.

In another embodiment of the present invention, the oligonucleotide formulation comprises an oligonucleotide which is a homopolymer sequence of C nucleotides.

In another embodiment of the present invention, the oligonucleotide formulation comprises an oligonucleotide having a polyAT sequence at least 10 nucleotides in length; a polyAC sequence at least 10 nucleotides in length; a polyAG sequence at least 10 nucleotides in length; a polyAU sequence at least 10 nucleotides in length; a polyAI sequence at least 10 nucleotides in length; a polyGC sequence at least 10 nucleotides in length; a polyGT sequence at least 10 nucleotides in length; a polyGU sequence at least 10 nucleotides in length; a polyGI sequence at least 10 nucleotides in length; a polyCT sequence at least 10 nucleotides in length; a polyCU sequence at least 10 nucleotides in length; a polyCI sequence at least 10 nucleotides in length; a polyTI sequence at least 10 nucleotides in length; a polyTU sequence at least 10 nucleotides in length; or a polyUI sequence at least 10 nucleotides in length.

In a further embodiment, the oligonucleotide formulation comprises an oligonucleotide having at least one phosphodiester linkage.

In a further embodiment, the oligonucleotide formulation comprises an oligonucleotide having at least one ribonucleotide.

Preferably, the oligonucleotide formulation comprises an oligonucleotide having at least one modification to its chemical structure, more preferably at least two different modifications to its chemical structure.

In accordance to the present invention, there is also provided an oligonucleotide formulation comprising an oligonucleotide having at least one sulfur modification.

Preferably, the oligonucleotide formulation comprises an oligonucleotide having at least one phosphorothioated linkage; at least one phosphorodithioated linkage; or at least one boranophosphate linkage.

In a further embodiment, the oligonucleotide formulation comprises an oligonucleotide having at least one sulfur modified nucleobase moiety such as one sulfur modified ribose moiety, one 2′ modification to the ribose moiety, one 2′-O alkyl modified ribose moiety, one 2′-O methyl modified ribose, one 2′-methoxyethyl modified ribose, or one 2′-FANA modified ribose.

In a further embodiment, the oligonucleotide formulation comprises an oligonucleotide having at least one methylphosphonate linkage.

In a further embodiment, the oligonucleotide formulation comprises an oligonucleotide having at least one portion consisting of glycol nucleic acid (GNA) with an acyclic propylene glycol phosphorothioate backbone.

In a further embodiment, the oligonucleotide formulation comprises an oligonucleotide having at least one locked nucleic acid portion.

In another embodiment, the oligonucleotide formulation comprises an oligonucleotide having at least one phosphorodiamidate morpholino portion.

In another embodiment, the oligonucleotide formulation comprises an oligonucleotide having at least one abasic nucleic acid.

In another embodiment, the oligonucleotide formulation comprises an oligonucleotide having at least one 3-carbon linker.

In a further embodiment, the oligonucleotide formulation comprises an oligonucleotide having a linker to form a concatemer of two or more oligonucleotide sequences.

According to the present invention, the oligonucleotide formulation of the present invention comprises an oligonucleotide linked or conjugated at one or more nucleotide residues, to a molecule modifying the characteristics of the oligonucleotide to obtain one or more characteristics selected from the group consisting of higher stability, lower serum interaction, higher cellular uptake, an improved ability to be formulated, a detectable signal, higher therapeutic activity, better pharmacokinetic properties, specific tissue distribution and lower toxicity.

In a further embodiment, the oligonucleotide formulation comprises an oligonucleotide linked or conjugated to a PEG molecule.

In a further embodiment, the oligonucleotide formulation comprises an oligonucleotide linked or conjugated to a cholesterol molecule.

In a further embodiment, the oligonucleotide formulation comprises a double stranded oligonucleotide.

In another embodiment, the oligonucleotide formulation comprises an oligonucleotide having at least one base which is capable of hybridizing via non-Watson-Crick interactions.

According to an embodiment of the present invention, the oligonucleotide formulation comprises an oligonucleotide having a portion complementary to a genome.

According to an embodiment of the present invention, the oligonucleotide formulation comprises an oligonucleotide binding to one or more cytokine protein.

In a further embodiment, the oligonucleotide formulation comprises an oligonucleotide that interacts with one or more cellular components, wherein said interaction resulting in inhibition of a protein activity or expression.

In one embodiment of the present invention, the oligonucleotide formulation comprises an oligonucleotide wherein at least a portion of the sequence of said oligonucleotide is derived from a genome. The oligonucleotide of the present invention has at most 90%, preferably 80%, more preferably 75% identity with the genomic sequence.

In accordance with the present invention, there is also provided an oligonucleotide formulation comprising an oligonucleotide that targets a disease wherein said target disease is dyslepidemia, cholesterol related condition, obesity, thrombotic disorder, osteoporosis, or snake venom effect.

In a further embodiment, the oligonucleotide mixture comprises a mixture of at least two different oligonucleotides. More preferably, the oligonucleotide formulation of the present invention comprises a mixture of at least ten different oligonucleotides or at least 100 different oligonucleotides; or at least 1000 different oligonucleotides; or at least 10⁶ different oligonucleotides.

In accordance with the present invention, there is also provided a therapeutic pharmaceutical composition comprising a therapeutically effective amount of at least one pharmacologically acceptable oligonucleotide formulation according to the present invention; and a pharmaceutically acceptable carrier. More preferably, the therapeutic pharmaceutical composition is adapted for the treatment, control, or prevention of a microbial infection disease.

In a further embodiment, the disease is dyslepidemia, cholesterol related condition, obesity, thrombotic disorder, osteoporosis, or snake venom effect.

In an additional embodiment, there is provided a therapeutic pharmaceutical composition according to the present invention, adapted for delivery by a mode selected from the group consisting of oral ingestion, inhalation, subcutaneous injection, intramuscular injection, intrathcal injection, intracerebral injection, by enema, skin topical administration and intravenous injection.

In a further embodiment, the therapeutic pharmaceutical composition further comprises a delivery system; at least one other therapeutic drug in combination; a non-nucleotidic therapeutic drug in combination; a therapeutic antisense, siRNA or sequence-specific aptamer oligonucleotide in combination; a therapeutic RNAi-inducing oligonucleotide.

In accordance to the present invention, there is also provided a method for the prophylaxis or treatment of a disease in a subject, comprising administering to a subject in need of such treatment a therapeutically effective amount of at least one pharmacologically acceptable oligonucleotide formulation according to the present invention, or therapeutic pharmaceutical composition according to any the present invention. More preferably, said disease is dyslepidemia, cholesterol related condition, obesity, thrombotic disorder, osteoporosis, or snake venom effect. In addition, said subject is a human or a non-human animal.

In accordance with the present invention, there is provided a use of a therapeutically effective amount of at least one pharmacologically acceptable oligonucleotide formulation according to the present invention, or therapeutic pharmaceutical composition according to the present invention for the prophylaxis or treatment of a disease in a subject. More preferably, said disease is dyslepidemia, cholesterol related condition, obesity, thrombotic disorder, osteoporosis, or snake venom effect. In addition, said subject is a human or a non-human animal

### DETAILED DESCRIPTION OF THE PREFERRED EMBODIMENT

The present invention is concerned with the identification and use of therapeutic ONs that act by a sequence independent mechanism of action, and includes the discovery that the therapeutic activity is greater for larger ONs and for ONs with a hydrophobic character such as sulfur modification and or a polyanionic nature.

ONs have been tested for a wide range of therapeutic activity as antisense. However, such antisense ONs are typically sequence-specific and target intracellular mRNA and typically are about 16-25 nucleotides in length.

As disclosed in the present invention, the therapeutic effect of randomer ONs is sequence independent. Considering the volumes and concentrations of ONs used in these assays, it is theoretically unlikely that a particular sequence is present at more than 1 copy in the mixture. This means than there can be no antisense or sequence-specific aptameric effect in these ONs randomers. In the present invention, should the diseases inhibition effect be caused by the sequence-specificity of the ONs, such effect would thus have to be caused by only one molecule, a result that does not appear possible. For example, for an ON randomer 40 bases in length, any particular sequence in the population would theoretically represent only ¼⁴⁰ or 1/8.27×10⁻²⁵ of the total fraction. Given that 1 mole=6.022×10²³ molecules, and the fact that our largest synthesis is currently done at the 15 micromole scale, all possible sequences will not be present and also, each sequence is present most probably as only one copy. Of course, one skilled in the art applying the teaching of the present invention could also use sequence specific ONs, but utilize the sequence independent activity discovered in the present invention.

In the context of the present invention, unless specifically limited or specified, the term “oligonucleotide (ON)” means oligodeoxynucleotide (ODN) or oligodeoxyribonucleotide or oligoribonucleotide. Thus, “oligonucleotide” refers to an oligomer or polymer of ribonucleic acid (RNA) and/or deoxyribonucleic acid (DNA) and/or analogs thereof. This term includes oligonucleotides composed of naturally-occurring nucleobases, sugars and covalent internucleoside (backbone) linkages as well as oligonucleotides having non-naturally-occurring portions. Examples of modifications that can be used are described herein. Oligonucleotides that include backbone and/or other modifications can also be referred to as oligonucleosides. Except otherwise specified, oligonucleotide definition includes homopolymers, heteropolymers, randomers (see below), random sequence oligonucleotides, genomic-derived sequence oligonucleotides and oligonucleotides purified from natural sources.

As used herein in connection with the action of a material, the phrase “sequence independent activity” or “sequence independent mode of action” indicates that the mechanism by which the material exhibits a therapeutic effect is not due to hybridization of complementary nucleic acid sequences, e.g., an antisense effect, and it is not due to a sequence-specific aptameric activity. Conversely, a “sequence dependant mode of action or activity” means that the therapeutic effect of a material involves hybridization of complementary nucleic acid sequences or involves a sequence-specific aptameric interaction.

As used herein the term “therapeutic” means treating, inhibiting, reverting, curing, or preventing a disease. A therapeutic agent means an agent that can be used for treating, inhibiting, reverting, curing, or preventing a disease.

As used herein the term “disease” means thrombotic disorder, cholesterol related disorder, dyslipidemia, osteoporosis or snake venom effect.

The term “thrombotic disorder” as used herein encompasses conditions associated with or resulting from thrombosis or a tendency towards thrombosis. These conditions include, without restriction, conditions associated with arterial thrombosis, such as coronary artery thrombosis and resulting myocardial infarction, cerebral artery thrombosis or intracardiac thrombosis (e.g., atrial fibrillation) and resulting stroke, and other peripheral arterial thromboses and occlusions; conditions associated with venous thrombosis, such as deep venous thrombosis and pulmonary embolisms; conditions associated with exposure of the patient's blood to a foreign or injured tissue surface, including diseased heart valves, mechanical heart valves, vascular grafts, and other extracorporeal devices such as intravascular cannulas and stents, vascular access shunts in hemodialysis patients, hemodialysis machines and cardiopulmonary by pass machines; conditions associated with coagulapathies, such as hypercoagulability and disseminated intravascular coagulopathy that are not the result of an endotoxin-initiated coagulation cascade; veno-occlusive disease (VOD); ischemia; and claudication.

The term “dyslipidemia” as used herein encompasses conditions associated with or resulting from a disorders in the lipoprotein or lipid metabolism including hypercholesterolemia, high triglycerides values (hypertriglyceridemia), hyperlipidemia, combined hyperlipidemia and increased level of free fatty acids. The term “dyslipidemia” as used herein also includes the metabolic syndrome, obesity and overweight.

The term “cholesterol related condition” as used herein encompasses conditions associated with or resulting from high level of, or a tendency towards, high level of cholesterol (hypercholelesterolemia) or of low density lipoproteins (LDL) or of very low density lipoproteins (VLDL) or of lipids in the blood or in any other organ or tissue. The term “cholesterol related condition” as used herein also includes abnormal level of an apolipoprotein in the blood or in any other organ or tissue. The term “cholesterol related condition” also includes artheriosclerosis characterized by the deposition of lipids, including cholesterol, in the arterial vessel wall.

The term “osteoporosis” as used herein encompasses conditions associated with or resulting from a decrease in bone mass and density or a tendency towards a decrease in bone mass and density.

The term “snake venom effect” as used herein encompasses conditions associated with or resulting from the contact of a subject with snake venom.

As used herein in connection with ONs or other materials, the term “therapeutic” refers to an effect due to the presence of ONs or other material in treating, inhibiting, stopping, reverting, curing or preventing a disease in cells, systems or organisms. In certain embodiments of the present invention, therapeutic ONs will have therapeutic activity against multiple diseases.

The term “therapeutic oligonucleotide formulation” refers to a preparation that includes at least one therapeutic oligonucleotide that is adapted for use as a therapeutic agent. The formulation includes the ON or ONs, and can contain other materials that do not interfere with their use as therapeutic agents in vivo. Such other materials can include without restriction diluents, excipients, carrier materials, delivery systems and/or other therapeutic materials.

As used herein, the term “pharmaceutical composition” refers to a therapeutic ON formulation that includes a physiologically or pharmaceutically acceptable carrier or excipient. Such compositions can also include other components that do not make the composition unsuitable for administration to a desired subject, e.g., a human.

As used in connection with a therapeutic formulation, pharmaceutical composition, or other material, the phrase “adapted for use as a therapeutic agent” indicates that the material exhibits a therapeutic effect and does not include any component or material that makes it unsuitable for use in inhibiting such disease in an in vivo system, e.g., for administering to a subject such as a human subject.

As used herein in connection with administration of a therapeutic material, the term “subject” refers to a living higher organism, including, for example, animals such as mammals, e.g., humans, non-human primates and non-human animals.

In the present invention, the term “randomer” is intended to mean a single stranded nucleic acid polymer, modified or not, having degenerate sequences at every position, such as NNNNNNNNNN. Each degenerate nucleotide position actually exists as a random population of the five naturally occurring bases on the nucleotide (adenine, guanine, cytosine, thymine, uracil) at this particular position, resulting in a completely degenerate pool of ONs of the same size but having no sequence identity as a population. Randomers can also include nucleobases which do not occur naturally including without restriction hypoxanthine, xanthosine, imidazole, 2-aminopurines or 5-nitroindole. The term randomer can apply to a sequence or a portion of a sequence.

In the present invention, the term degenerate means that a sequence is made of a mix of nucleotides. A completely degenerate sequence means that A, C, G, and T (or other nucleobases) are randomly used at each position of the sequence and nucleotide position are identified by N (see randomer definition). A degenerate sequence means also that at least two nucleobases are randomely used at each position of the sequence. Degenerate can apply to a sequence, a portion of a sequence or one nucleotide position in a sequence.

As used herein, the term “delivery system” refers to a component or components that, when combined with an ON as described herein, facilitates the transfer of ONs inside cells or increases the amount of ONs that contact the intended location in vivo, and/or extends the duration of its presence at the target or increases its circulating lifetime in vivo, e.g., by at least 10, 20, 50, or 100%, or even more as compared to the amount and/or duration in the absence of the delivery system. The term delivery system also means encapsulation system or encapsulation reagent. To encapsulate ONs means to put in contact an ON with a delivery system or an encapsulation reagent. An ON in contact with a delivery system can be referred to as an “encapsulated ON”.

The term “therapeutically effective amount” refers to an amount that is sufficient to effect a therapeutically or prophylactically significant reduction of diseases when administered to a typical subject of the intended type. In aspects involving administration of a therapeutic ON to a subject, typically the ON, formulation, or composition should be administered in a therapeutically effective amount.

According to the data reported herein, ONs have potential therapeutic activity against thrombotic disorders. Therefore to test this hypothesis, sulfur modified ONs were selected to be tested for binding to proteins known to be involved in such diseases. We observed that degenerate PS-ONs interact with thrombin and fibrinogen and that this interaction is sequence independent, size dependent and dependent on chemical (i.e. sulfur) modification. Consequently, the present invention discloses that ONs can have a therapeutic activity by binding to thrombin or fibrinogen involved in thrombotic disorders or to other proteins and receptors involved in thrombotic disorders and therefore preventing, inhibiting or reversing thrombotic disorders.

According the data reported herein, ONs have potential therapeutic activity against cholesterol related conditions, dyslipidemia, the metabolic syndrome and obesity. Therefore to test this hypothesis, sulfur modified ONs were selected to be tested for binding to proteins known to be involved in such diseases. We observed that ONs interact with various proteins involved in cholesterol related conditions, dyslipidemia, the metabolic syndrome and obesity such as apolipoprotein B, apolipoprotein E, LDL, VLDL, resistin, TNF-alpha, IL-1 and IL-6. This type of protein-ON interaction is sequence independent, size dependent and dependent on chemical (i.e. sulfur) modification. Consequently, the present invention discloses that ONs can have a therapeutic activity by binding to proteins involved in cholesterol related conditions, dyslipidemia, the metabolic syndrome, obesity and other related conditions and disorders and therefore preventing, inhibiting or reversing such conditions and disorders.

According to the following discussion, ONs have potential therapeutic activity against osteoporosis. It was shown previously that ONs are membrane fusion inhibitors (Vaillant et al., 2006, Antimicrob. Agents Chemother. 50: 393-1401) in many viral infections. In viral fusion membranes from the virus and the host cell are brought in to close proximity by fusion proteins leading to membrane fusion. ONs prevent this viral fusion through a sequence independent, size dependent and chemistry dependent (i.e. sulfur modification) mode of action. Similar to viral fusion, osteoclast formation is due to syncytia formed after the fusion of mononuclear phagocytes. The multinucleated osteoclast is produced by cell membrane fusion. It was then rationalize that ONs may also inhibit osteoclast formation by at least one mechanism of action with similar chemical determinants of activity as seen for inhibition of viral fusion. ONs may have a therapeutic activity against cell fusion implicated in the formation of osteoclasts which are involved in bone resorption and responsible for osteoporosis. Therefore ONs could be used for preventing, inhibiting or reversing osteoporosis.

According to the present invention, ONs have potential therapeutic activity against snake venom activity. Therefore to test this hypothesis, sulfur modified ONs were selected to be tested for binding to snake venoms. It was observed that degenerate PS-ONs interact with snake venoms and that this interaction is sequence independent, size dependent and effective with a sulfur modification to the backbone. Thus, the present invention discloses that ONs can have a therapeutic activity by binding to snake venoms involved in snake venom effects and therefore preventing, inhibiting or reversing such effects.

According to the present invention, ONs have the capacity to treat animals, including humans, suffering from a disease consisting of thrombotic disorders, cholesterol and dyslipidemia disorders, osteoporosis and snake venom. Therefore to test this hypothesis, a sulfur modified ON was selected to be tested in a hypercholesterolemia/obesity/dyslipidemia in vivo model. Results show that ON administration resulted in inhibition of weight gain, of body fat accumulation, of increase in triglycerides and of hypercholesterolemia. In addition, ONs can be used as therapeutic agent or in method of treatment for a disease, for example but without restriction, dyslipidemia, hypercholesterolemia, hypertriglyceridemia, obesity and the metabolic syndrome.

One skilled in the art applying the teaching of the present invention could also use ONs with different chemical modifications. A modification of the ON, such as, but not limited to, a phosphorothioate (PS) modification or other sulfur modifications, appears to be beneficial for therapeutic activity. Such sulfur modifications may include without restriction mono and diphosphorothioation of the phosphodiester linkage, 4- or 5-thiolation of the uridine moiety, 5-thiolation of the cytidine moiety, 2- or 4-thiolation of the thymine moiety, 6-thiolation of the guanine moiety, sulfur modifications to any other nucleobase moiety and sulfur modifications to the ribose moiety of any nucleotide or combinations of any of the above mentioned modifications. Moreover, ONs may have more than one sulfur substitution on each nucleotide, which can potentially increase the activity. Finally, any single or multiple sulfur substitution may be combined with other modifications known to improve properties of ONs. ONs of this invention may also have chemical modifications including without restriction: any 2′ ribose modification including 2′-O methyl, 2′-fluorine, 2′-FANA, 2′-methoxyethyl, locked nucleic acids, methylphosphonates, boranophosphates and phosphorodiamidate morpholino oligomers. Moreover ONs may have a structure of or comprise a portion consisting of glycol nucleic acid (GNA) with an acyclic propylene glycol phosphodiester backbone capable of forming stable antiparallel duplexes following the Watson-Crick base pairing rules (Zhang et al., 2005, J. Am. Chem. Soc. 127(12): 4174-4175). Such GNAs may comprise phosphorothioate linkages or other appropriate modifications as described above. Additionally, oligos may include one or more abasic or 3-carbon linkers, which can serve to remove either the base or the base and the sugar moieties from the phosphorothioate backbone.

One aspect of the invention provides a therapeutic ON targeting a disease. Such an ON comprises at least one active ON and is adapted for use as a therapeutic agent.

Another aspect of the invention provides a formulation including a therapeutic ON targeting a disease. Such an ON comprises at least one active ON and is adapted for use as a therapeutic agent.

In another aspect, ONs of this invention may be in the form of a formulation targeting thrombin or fibrinogen involved in diseases. Such a formulation comprises at least one active ON and is adapted for use as a therapeutic agent.

In another aspect, ONs of this invention may be in the form of a formulation targeting Apo B, Apo E, other apolipoproteins, LDL, VLDL, resistin, TNF-alpha, IL-1, and IL-6 involved in diseases. Such a formulation comprises at least one active ON and is adapted for use as a therapeutic agent.

In another aspect, ONs of this invention may be in the form of a formulation targeting osteoclast formation involved in diseases. Such a formulation comprises at least one active ON and is adapted for use as a therapeutic agent.

In another aspect, ONs of this invention may be in the form of a formulation targeting snake venom involved in diseases. Such a formulation comprises at least one active ON and is adapted for use as a therapeutic agent.

In another aspect, the ONs of this invention may be in the form of a pharmaceutical composition useful for treating (or prophylaxis of) a disease, which may be approved by a regulatory agency for use in humans or in non-human animals, and/or against a particular disease. Such a pharmaceutical composition comprises at least one therapeutically active ON and is adapted for use as a therapeutic agent. This pharmaceutical composition may include physiologically and/or pharmaceutically acceptable carriers. The characteristics of the carrier may depend on the route of administration. The pharmaceutical composition of the invention may also contain other active factors and/or agents which enhance activity.

In yet another aspect, the invention provides a method for the prophylaxis or treatment of a disease in a subject by administering to a subject in need of such treatment a therapeutically effective amount of at least one pharmacologically acceptable ON as described herein, e.g., a sequence independent ON at least 6 nucleotides in length, more preferably 10 nucleotides in length, or a pharmaceutical composition or formulation containing such ON. In particular embodiments, the disease is related to a disease or condition indicated herein; the subject is a type of subject as indicated herein, e.g., human, non-human animal, non-human mammal, bird, fish and the like.

In a particular embodiment, the therapeutic ON, ON formulation, ON pharmaceutical composition or ON method of treatment described herein prevent, reverse or inhibit thrombin or plasminogen activity involved in thrombotic disorders; or Apo B, Apo E, other apolipoproteins, LDL, VLDL, resistin, TNF-alpha, IL-1, and IL-6 involved in cholesterol related disorders, dyslipidemia, the metabolic syndrome or obesity; or cell fusion leading to osteoclasts involved in osteroporosis; or snake venom involved in snake venom effects.

In a yet another embodiment of the present invention, the therapeutic ON, ON formulation, ON pharmaceutical composition or method of treatment described herein may be administered therapeutically or prophylactically to treat a disease.

In particular embodiments, the disease targeted by ONs, formulations thereof, pharmaceutical compositions thereof or methods of treatment thereof can be a thrombotic disorder such as described in the following: an increased tendency toward thrombosis accompanies surgery, trauma, many inflammatory disorders, malignancy, pregnancy, obesity, vascular disorders and prolonged immobilization. Inherited thrombotic tendencies, which are much rarer, are being increasingly recognized and include deficiencies of the protein C-protein S system, deficiencies of antithrombin III (ATIII), dysfibrinogenemias, and other disorders of the fibrinolytic system. Severe derangements of the coagulation process are seen in disseminated intravascular coagulation (DIC), a syndrome characterized by the slow formation of fibrin microthrombi in the microcirculation and the development of concomitant fibrinolysis. These primary disorders fall into three general categories: (1) release of procoagulant substances into the blood, as may occur in amniotic fluid embolism, abruptio placentae, certain snake bites, and various malignancies, (2) contact of blood with an injured or abnormal surface, as may occur in extensive burns, infections, heat stroke, organ grafts, and during extracorporal circulation, and (3) generation of procoagulant-active substances within the blood, as may occur if red or white blood cell or platelet membranes become damaged and release thromboplastic substances, e.g., during leukemia treatment, hemolytic transfusion reactions and microangiopathic hemolytic anemia. Bacterial endotoxins associated with or released from gram-negative bacteria also have thromboplastin-like properties that initiate clotting. Intravascular clotting occurs most frequently with shock, sepsis, cancer, obstetric complications, burns, and liver disease.

In a yet another particular embodiment, the therapeutic ON, ON formulation, ON pharmaceutical composition or method of treatment described herein may be administered therapeutically or prophylactically to treat thrombotic disorder associated with fibrinogen or thrombin activity. The ONs of the invention may act to ameliorate the course of thrombotic disorders by mechanisms including, without limitation, interference with thrombin enzymatic activity or interference with the cleavage of fibrinogen leading to fibrin production or interference with fibrin deposition.

In particular embodiments, the disease targeted by ONs, formulations thereof, pharmaceutical compositions thereof or methods of treatment thereof can be a cholesterol related condition such as described in the following: hypercholesterimia, atherosclerosis, high level of LDL, high level of VLDL, high level of apolipoproteins, cerebral atherosclerosis, cholesteryl ester storage disorder

In particular embodiments, the disease targeted by ONs, formulations thereof, pharmaceutical compositions thereof or methods of treatment thereof can be dyslipidemia such as described in the following: hyperlipidemia, hypercholesterimia, hyperlipoproteinemia, hypertryglyceridemia, the metabolic syndrome, obesity and overweight

In particular embodiments, the disease targeted by ONs, formulations thereof, pharmaceutical compositions thereof or methods of treatment thereof can be osteoporosis or snake venom effect.

The present invention involves the discovery that oligonucleotides (ONs), e.g., oligodeoxynucleotides (ODNs), including modified oligonucleotides, can have a therapeutic application through a sequence independent mode of action. It is not necessary for the oligonucleotide to be complementary to any sequence or to have a particular distribution of nucleotides in order to have activity. Such an oligonucleotide can even be prepared as a randomer, such that there will be at most a few copies of any particular sequence in a preparation, e.g., in a 15 micromol randomer preparation 32 or more nucleotides in length.

In addition, it is disclosed that different length oligonucleotides have different activity. For example, present results indicate that the length of oligonucleotide that produces maximal effect when modified with sulfur linkages is typically in the range of 30-120 nucleotides but not restricted to these length. In view of the present discoveries concerning properties of oligonucleotides, this invention provides oligonucleotide agents that can have activity against diseases and conditions described herein. Such agents are particularly advantageous in view of the limited therapeutic options currently available.

Therefore, the ONs, e.g., ODNs, of the present invention are useful in therapy for treating or preventing diseases and conditions described herein. Such treatments are applicable to many types of patients and treatments, including, for example, the prophylaxis or treatment of diseases and conditions described herein.

A first aspect of the invention concerns oligonucleotides, e.g., purified oligonucleotides, where the activity occurs principally by a sequence independent (e.g., non-sequence complementary or non-sequence dependant aptameric activity) mode of action, and formulations containing such oligonucleotides.

Oligonucleotides useful in the present invention can be of various lengths, e.g., at least 6, more preferably 10, 14, 15, 20, 21, 22, 23, 24, 25, 26, 27, 28, 29, 30, 31, 32, 33, 34, 35, 36, 37, 38, 39, 40, 41, 42, 43, 44, 45, 50, 60, 70, 80, 90, 100, 110, 120, 140, 160, or more nucleotides in length. Likewise, the oligonucleotide can be in a range, e.g., a range defined by taking any two of the preceding listed values as inclusive end points of the range, for example 10-20, 20-30, 20-40, 30-40, 30-50, 40-50, 40-60, 40-80, 50-60, 50-70, 60-70, 70-80, 60-120, and 80-120 nucleotides. In particular embodiments, a minimum length or length range is combined with any other of the oligonucleotide specifications listed herein for the present oligonucleotides.

The nucleotide can include various modifications, e.g., stabilizing modifications, and thus can include at least one modification in the phosphodiester linkage and/or on the sugar, and/or on the base. For example, the oligonucleotide can include one or more phosphorothioate linkages, phosphorodithioate linkages, and/or methylphosphonate linkages. Different chemically compatible modified linkages can be combined, e.g., modifications where the synthesis conditions are chemically compatible. While modified linkages are useful, the oligonucleotides can include phosphodiester linkages, e.g., include at least one phosphodiester linkage, or at least 5, 10, 20, 30% or more phosphodiester linkages. Additional useful modifications include, without restriction, modifications at the 2′-position of the sugar, such as 2′-O-alkyl modifications such as 2′-O-methyl modifications, 2′-amino modifications, 2′-halo modifications such as 2′-fluoro; acyclic nucleotide analogs. Other modifications are also known in the art and can be used. In particular embodiments, the oligonucleotide has modified linkages throughout, e.g., phosphorothioate; has a 3′- and/or 5′-cap; includes a terminal 3′-5′ linkage; the oligonucleotide is or includes a concatemer consisting of two or more oligonucleotide sequences joined by a linker(s).

The present invention further provides an oligonucleotide, wherein said oligonucleotide is linked or conjugated at one or more nucleotide residues, to a molecule modifying the characteristics of the oligonucleotide to obtain one or more characteristics selected from the group consisting of higher stability, lower serum interaction, higher cellular uptake, higher protein interaction, an improved ability to be formulated for delivery, a detectable signal, higher activity, better pharmacokinetic properties, specific tissue distribution, lower toxicity.

In certain embodiments, the oligonucleotide includes at least 10, 20, 30, 40, 50, 60, 70, 80, 90, 95, or 100% modified linkages, e.g., phosphorothioate, phosphorodithioate, and/or methylphosphonate.

In certain embodiments, at least 10, 20, 30, 40, 50, 60, 70, 80, 90, or 95%, or all of the nucleotides are modified at the 2′-position of the ribose, e.g., 2′-OMe, 2′-F, 2′-amino.

In certain embodiments modified linkages are combined with 2′-modifications in oligonucleotides, for example, at least 30% modified linkages and at least 30% 2′-modifications; or respectively at least 40% and 40%, at least 50% and 50%, at least 60% and 60%, at least 70% and 70%, at least 80% and 80%, at least 90% and 90%, 100% and 100%. In certain embodiments, the oligonucleotide includes at least 30, 40, 50, 60, 70, 80, 90, or 100% modified linkages and at least 30, 40, 50, 60, 70, 80, 90, or 100% 2′-modifications where embodiments include each combination of listed modified linkage percentage and 2′-modification percentage (e.g., at least 50% modified linkage and at least 80% 2′-modifications, and at least 80% modified linkages and 100% 2′-modifications). In particular embodiments of each of the combinations percentages described, the modified linkages are phosphorothioate linkages; the modified linkages are phosphorodithioate linkages; the 2′-modifications are 2′-OMe; the 2′-modifications are 2′-fluoro; the 2′-modifications are a combination of 2′-OMe and 2′-fluoro; the modified linkages are phosphorothioate linkages and the 2′-modifications are 2′-OMe; the modified linkages are phosphorothioate linkages and the 2′-modifications are 2′-fluoro; the modified linkages are phosphorodithioate linkages and the 2′-modifications are 2′-OMe; the modified linkages are phosphorodithioate linkages and the 2′-modifications are 2′-fluoro; the modified linkages are phosphorodithioate linkages and the 2′-modifications are a combination of 2′-OMe and 2′-fluoro. In certain embodiments of oligonucleotides as described herein that combine a particular percentage of modified linkages and a particular percentage of 2′-modifications, the oligonucleotide is at least 15, 20, 25, 30, 35, 40, 50, 60, 70, 80, 90, 100, 110, or 120 nucleotides in length, or is in a length range defined by taking any two of the specified lengths as inclusive endpoints of the range.

In certain embodiments, all but 1, 2, 3, 4, 5, 6, 7, 8, 9, or 10 of the internucleotidic linkages and/or 2′-positions of the ribose moiety are modified, e.g., with linkages modified with phosphorothioate, phosphorodithioate, or methylphosphonate linkages and/or 2′-OMe, 2′-F, and/or 2′-amino modifications of the ribose moiety.

In some embodiments, the oligonucleotide includes at least 1, 2, 3, or 4 ribonucleotides, or at least 10, 20, 30, 40, 50, 60, 70, 80, 90%, or even 100% ribonucleotides.

In particular embodiments, the oligonucleotide includes non-nucleotide groups in the chain (i.e., form part of the chain backbone) and/or as side chain moieties, e.g., 1, 2, 3, 4, 5, 6, 7, 8, 9, 10, or even more, or up to 5, 10, 20% or more of the chain moieties and/or side chain moieties.

In certain embodiments, the oligonucleotide is free of self-complementary sequences longer than 5, 8, 10, 15, 20, 25, 30 nucleotides; the oligonucleotide is free of catalytic activity, e.g., cleavage activity against RNA; the oligonucleotide does not induce an RNAi mechanism.

In particular embodiments, the oligonucleotide binds protein involved in a disease or condition described in the present invention; the sequence of the oligonucleotide (or a portion thereof, e.g., at least 20, 30, 40, 50, 60, 70% or more) is derived from a genome; the activity of an oligonucleotide with a sequence derived from a genome is not superior to a randomer oligonucleotide or a random oligonucleotide of the same length; the oligonucleotide includes a portion complementary to a genome sequence and a portion not complementary to a genome sequence; unless otherwise indicated, the sequence of the oligonucleotide includes A(x), C(x), G(x), T(x), U(x), I(x), AC(x), AG(x), AT(x), AU(x), CG(x), CT(x), CU(x), GT(x), GU(x), TU(x), AI(x), IC(x), IG(x), IT(x) IU(x) where x is 2, 3, 4, 5, 6, . . . 60 . . . 120 (in particular embodiments the oligonucleotide is at least 15, 20, 25, 29, 30, 32, 34, 35, 36, 38, 40, 45, 46, 50, 60, 70, 80, 90, 100, 110, 120, 140, or 160 nucleotides in length or is in a range defined by taking any two of the listed values as inclusive endpoints, or the length of the specified repeat sequence is at least a length or in a length range just specified); the oligonucleotide includes a combination of repeat sequences (e.g., repeat sequences as specified above), including, for example, each combination of the above monomer and/or dimer repeats taken 2, 3, or 4 at a time; the oligonucleotide is single stranded (RNA or DNA); the oligonucleotide is double stranded (RNA or DNA); the oligonucleotide includes at least one Gquartet or CpG portion; the oligonucleotide includes a portion complementary to a mRNA and is at least 29, 37, or 38 nucleotides in length (or other length as specified above); the oligonucleotide includes at least one non-Watson-Crick oligonucleotide and/or at least one nucleotide that participates in non-Watson-Crick binding with another nucleotide and/or at least one nucleotide that cannot form base pairs with other nucleotides; the oligonucleotide is a random oligonucleotide, the oligonucleotide is a randomer or includes a randomer portion, e.g., a randomer portion that has a length of at least 5, 10, 15, 20, 25, 30, 35, 40 or more contiguous oligonucleotides or a length as specified above for oligonucleotide length or at least 10, 20, 30, 40, 50, 60, 70, 80, 90% or all the nucleotides are randomer; the oligonucleotide is linked or conjugated at one or more nucleotide residues to a molecule that modifies the characteristics of the oligonucleotide, e.g. to provide higher stability (such as stability in serum or stability in a particular solution), lower serum interaction, higher cellular uptake, higher protein interaction, improved ability to be formulated for delivery, a detectable signal, improved pharmacokinetic properties, specific tissue distribution, and/or lower toxicity.

It was also discovered that phosphorothioated ONs containing only (or at least primarily) pyrimidine nucleotides, including cytosine and/or thymidine and/or other pyrimidines are resistant to low pH and polycytosine oligonucleotides showed increased resistance to a number of nucleases, thereby providing two important characteristics for oral administration of an ON. Thus, in certain embodiments, the oligonucleotide has at least 80, 90, or 95, or 100% modified internucleotidic linkages (e.g., phosphorothioate or phosphorodithoiate) and the pyrimidine content is more than 50%, more than 60%, more than 70%, more than 80%, more than 90%, or 100%, i.e.; is a pyrimidine oligonucleotide or the cytosine content is more than 50%, more than 60%, more than 70%, more than 80%, more than 90% or 100% i.e. is a polycytosine oligonucleotide. In certain embodiments, the length is at least 29, 30, 32, 34, 36, 38, 40, 45, 50, 60, 70, or 80 nucleotides, or is in a range of 20-28, 25-35, 29-40, 30-40, 35-45, 40-50, 45-55, 50-60, 55-65, 60-70, 65-75, or 70-80, or is in a range defined by taking any two of the listed values as inclusive endpoints of the range. In particular embodiment, the oligonucleotide is at least 50, 60, 70, 80, or 90% cytosine; at least 50, 60, 70, 80, or 90% thymidine (and may have a total pyrimidine content as listed above). In particular embodiments, the oligonucleotide contains a listed percentage of either cytosine or thymidine, and the remainder of the pyrimidine nucleotides are the other of cytosine and thymidine. Also in certain embodiments, the oligonucleotide includes at least 10, 12, 14, 16, 18, 20, 25, 30, 35, 40, or more contiguous pyrimidine nucleotides, e.g., as C nucleotides, T nucleotides, or CT dinucleotide pairs. In certain embodiments, the pyrimidine oligonucleotide consists only of pyrimidine nucleotides; includes at least 1, 2, 3, 4, 5, 6, 7, 8, 9, or 10 non-pyrimidine moieties; includes 1-5, 6-10, 11-15, or at least 16 non-pyrimidine backbone moieties; includes at least one, 1-20, 1-5, 6-10, 11-15, or 16-20 non-nucleotide moieties; includes at least one, 1-20, 1-5, 6-10, 11-15, or 16-20 purine nucleotides. Preferably, in embodiments in which non-nucleotide moieties are present, the linkages between such moieties or between such moieties and nucleotides are at least 25, 35, 50, 70, 90, or 100% as resistant to acidic conditions as PS linkages in a 40-mer polyC oligonucleotide as evaluated by gel electrophoresis under conditions appropriate for the size and chemistry of the oligonucleotide.

Oligonucleotides can also be used in combinations, e.g., as a mixture. Such combinations or mixtures can include, for example, at least 2, 3, 4, 5, 10, 20, 50, 100, 1000, 10000, 100,000, 1,000,000, or more different oligonucleotides, e.g., any combination of oligonucleotides are described herein. Such combinations or mixtures can, for example, be different sequences and/or different lengths and/or different modifications and/or different linked or conjugated molecules. In particular embodiments of such combinations or mixtures, a plurality of oligonucleotides have a minimum length or are in a length range as specified above for oligonucleotides. In particular embodiments of such combinations or mixtures, at least one, a plurality, or each of the oligonucleotides can have any of the other properties specified herein for individual oligonucleotides (which can also be in any consistent combination).

In certain embodiments, the sequence of the oligonucleotide is not perfectly complementary to any equal length portion of the a genome sequence, or has less than 95, 90, 80, 70, 60, or 50% complementarity to any equal length portion of the genomic sequence, the oligonucleotide sequence does not consist essentially of polyA, polyC, polyG, polyT, Gquartet, or a TG-rich sequence.

As used in connection with the present oligos, the term “TG-rich” indicates that the sequence of the oligonucleotide consists of at least 50 percent T and G nucleotides, or if so specified, at least 60, 70, 80, 90, or 95% T and G, or even 100%.

In a related aspect, the invention provides a mixture of oligonucleotides that includes at least two different oligonucleotides as described herein, e.g., at least 2, 3, 4, 5, 7, 10, 50, 100, 1000, 10,000, 100,000, 1,000,000, or even more.

Specification of particular lengths for oligonucleotides, e.g., at least 20 nucleotides in length, means that the oligonucleotide includes at least 20 linked nucleotides. Unless clearly indicated to the contrary, the oligonucleotide may also include additional, non-nucleotide moieties, which may form part of the backbone of the oligonucleotide chain. Unless otherwise indicated, when non-nucleotide moieties are present in the backbone, at least 10 of the linked nucleotides are contiguous.

As used herein in connection with the action of an oligonucleotide, “sequence independent mode of action” indicates that the particular biological activity is not dependent on a particular oligonucleotide sequence in the oligonucleotide. For example, the activity does not depend on sequence dependent hybridization such as with antisense activity, or a particular sequence resulting in a sequence dependent aptameric interaction. Similarly, the phrase “non-sequence complementary mode of action” indicates that the mechanism by which the material exhibits an effect is not due to hybridization of complementary nucleic acid sequences, e.g., an antisense effect. Conversely, a “sequence complementary mode of action” means that the effect of a material involves hybridization of complementary nucleic acid sequences or sequence specific aptameric interaction. Thus, indicating that the activity of a material is due to a sequence independent mode of action” or that the activity is “not primarily due to a sequence complementary mode of action” means that the activity of the oligonucleotide satisfies at least one of the 2 tests provided herein (see Examples). In particular embodiments, the oligonucleotide satisfies test 1 and test 2.

A related aspect concerns an oligonucleotide randomer or randomer formulation that contains at least one randomer, where the activity of the randomer occurs principally by a sequence independent, e.g., non-sequence complementary mode of action. Such a randomer formulation can, for example, include a mixture of randomers of different lengths, e.g., at least 2, 3, 5, 10, or more different lengths, or other mixtures as described herein.

The phrase “derived from a genome” indicates that a particular sequence has a nucleotide base sequence that has at least 70% identity to a genomic nucleotide sequence or its complement (e.g., is the same as or complementary to a genomic sequence), or is a corresponding RNA sequence. In particular embodiments of the present invention, the term indicates that the sequence is at least 70% identical to a genomic sequence of a particular gene involved in a disease or condition against which the oligonucleotide is directed, or to its complementary sequence. In particular embodiments, the identity is at least 80, 90, 95, 98, 99, or 100%. Genome can be from an animal, e.g. a human, from a microorganism, e.g. a virus, a bacteria, a parasite, or from a plant.

The invention also provides an pharmaceutical composition that includes a therapeutically effective amount of a pharmacologically acceptable, oligonucleotide or mixture of oligonucleotides as described herein, e.g., at least 6 nucleotides in length or other length as listed herein, where the activity of the oligonucleotide occurs principally by a sequence independent, e.g., non-sequence complementary or non-sequence dependent aptamer, mode of action, and a pharmaceutically acceptable carrier. In particular embodiments, the oligonucleotide or a combination or mixture of oligonucleotides is as specified above for individual oligonucleotides or combinations or mixtures of oligonucleotides. In particular embodiments, the pharmaceutical compositions are approved for administration to a human, or a non-human animal such as a non-human primate.

In particular embodiments, the pharmaceutical composition can be formulated for delivery by a mode selected from the group consisting of but not restricted to oral ingestion, oral mucosal delivery, intranasal drops or spray, intraocular injection, subconjonctival injection, eye drops, ear drops, by inhalation, intratracheal injection or spray, intrabronchial injection or spray, intrapleural injection, intraperitoneal injection perfusion or irrigation, intrathecal injection or perfusion, intracranial injection or perfusion, intramuscular injection, intravenous injection or perfusion, intraarterial injection or perfusion, intralymphatic injection or perfusion, subcutaneous injection or perfusion, intradermal injection, topical skin application, by organ perfusion, by topical application during surgery, intratumoral injection, topical application, gastric injection perfusion or irrigation, enteral injection or perfusion, colonic injection perfusion or irrigation, rectal injection perfusion or irrigation, by rectal suppository or enema, by urethral suppository or injection, intravesical injection perfusion or irrigation, or intraarticular injection.

In particular embodiments, the composition includes a delivery system, e.g., targeted to specific cells or tissues; a liposomal formulation, another drug, e.g., a non-nucleotide polymer, an antisense molecule, a siRNA, or a small molecule drug.

In particular embodiments, the oligonucleotide, oligonucleotide preparation, oligonucleotide formulation, or pharmaceutical composition has an in vitro IC₅₀ or EC₅₀ of 10, 5, 2, 1, 0.50, 0.20, 0.10, 0.09, 0.08, 0.07, 0.75, 0.06, 0.05, 0.045, 0.04, 0.035, 0.03, 0.025, 0.02, 0.015, or 0.01 μM or less.

In particular embodiments, the pharmaceutical composition contains at least one polypyrimidine oligonucleotide as described herein. In view of the resistance to low pH discovered for polypyrimidine oligonucleotides; in certain embodiments such a composition is adapted for delivery to an acidic in vivo site, e.g., oral delivery or vaginal delivery.

As used in relation to in vivo administration of the present oligonucleotides and compositions, the term “acidic site” means a site that has a pH of less than 7. Examples include the stomach (pH generally 1-2), the vagina (pH generally 4-5 but may be lower), and to a lesser degree, the skin (pH generally 4-6).

As used herein, the phrase “adapted for oral delivery” and like terms indicate that the composition is sufficiently resistant to acidic pH to allow oral administration without a clinically excessive loss of activity, e.g., an excessive first pass loss due to stomach acidity of less than 50% (or is indicated, less than 40%, 30%, 20%, 10%, or 5%).

As used herein in connection with agents and drugs or test compounds, the term “small molecule” means that the molecular weight of the molecule is 1500 daltons or less. In some cases, the molecular weight is 1000, 800, 600, 500, or 400 daltons or less.

In another aspect, the invention provides a kit that includes at least one oligonucleotide, oligonucleotide mixture, oligonucleotide formulation, or pharmaceutical composition that includes such oligonucleotide, oligonucleotide mixture, or oligonucleotide formulation in a labeled package, where the activity of the oligonucleotide occurs principally by a sequence independent e.g., non-sequence complementary or non-sequence dependent aptameric, mode of action and the label on the package indicates that the oligonucleotide can be used against at least one disease or condition.

In particular embodiments the kit includes a pharmaceutical composition that includes at least one oligonucleotide as described herein. In one embodiment, the kit contains a mixture of at least two different oligonucleotides. In one embodiment, the oligonucleotide is adapted for in vivo use in an animal and/or the label indicates that the oligonucleotide or composition is acceptable and/or approved for use in an animal; the animal is a mammal, such as human, or a non-human mammal such as bovine, porcine, a ruminant, ovine, or equine; the animal is a non-human animal; the animal is a bird, the kit is approved by a regulatory agency such as the U.S. Food and Drug Administration or equivalent agency for use in an animal, e.g., a human.

In particular embodiments, the different random oligonucleotides comprises randomers of different lengths; the random oligonucleotides can have different sequences or can have sequence in common, such as the sequence of the shortest oligos of the plurality; and/or the different random oligonucleotides comprise a plurality of oligonucleotides comprising a randomer segment at least 5 nucleotides in length or the different random oligonucleotides include a plurality of randomers of different lengths. Other oligonucleotides, e.g., as described herein oligonucleotides, can be tested in a particular system.

In yet another aspect, the invention provides a method for the prophylaxis or treatment in a subject by administering to a subject in need of such treatment a therapeutically effective amount of at least one pharmacologically acceptable oligonucleotide as described herein, e.g., a sequence independent oligonucleotide at least 6 nucleotides in length, or an pharmaceutical composition or formulation or mixture containing such oligonucleotide(s). In a further embodiment, the invention provides a use for the prophylaxis or treatment in a subject by administering to a subject in need of such treatment a therapeutically effective amount of at least one pharmacologically acceptable oligonucleotide as described herein, e.g., a sequence independent oligonucleotide at least 6 nucleotides in length, or an pharmaceutical composition or formulation or mixture containing such oligonucleotide(s). In particular embodiments, the disease or condition targeted be any of those listed herein as suitable for inhibition using the present invention; the subject is a type of subject as indicated herein, e.g., human, non-human animal, non-human mammal, bird, plant, and the like; the treatment is for a condition or a disease as indicated in the Background section herein.

In yet another aspect, the invention provides a method for the prophylaxis or treatment of a in an acidic environment in a subject, comprising administering to a subject in need of such a treatment a therapeutically effective amount of at least one pharmacologically acceptable pharmaceutical composition of the invention, said composition being adapted for administration to an acidic in vivo site.

In yet another aspect, the invention provides a use for the prophylaxis or treatment in an acidic environment in a subject, comprising administering to a subject in need of such a treatment a therapeutically effective amount of at least one pharmacologically acceptable pharmaceutical composition of the invention, said composition being adapted for administration to an acidic in vivo site.

In particular embodiments, the oligonucleotide is a polypyrimidine oligonucleotide (or a formulation or pharmaceutical composition containing such polypyrimidine oligonucleotide), which may be adapted for oral or vaginal administration, e.g., as described herein.

In particular embodiments, the oligonucleotide(s) is as described herein for the present invention, e.g., having a length as described herein; a method of administration as described herein is used; a use as described herein is used; a delivery system as described herein is used.

The term “therapeutically effective amount” refers to an amount that is sufficient to effect a therapeutically or prophylactically significant reduction of a disease or condition when administered to a typical subject of the intended type. In aspects involving administration of an oligonucleotide to a subject, typically the oligonucleotide, formulation, or composition should be administered in a therapeutically effective amount.

In certain embodiments involving oligonucleotide formulations, pharmaceutical compositions, treatment and prophylactic methods and/or treatment and prophylactic uses described herein, the oligonucleotide(s) having a sequence independent mode of action is not associated with a transfection agent; the oligonucleotide(s) having a sequence independent mode of action is not encapsulated in liposomes and/or non-liposomal lipid particles. In certain embodiments, the oligonucleotide(s) having a sequence independent mode of action is in a pharmaceutical composition or is administered in conjunction with (concurrently or sequentially) an oligonucleotide that acts principally by a sequence dependent mode of action, e.g., antisense oligonucleotide or siRNA, where the oligonucleotide(s) having a sequence dependent mode of action can be associated with a transfection agent and/or encapsulated in liposomes and/or non-liposomal lipid particles.

In yet another aspect, the invention provides a polymer mix that includes at least one oligonucleotide and at least one non-nucleotide polymer. In particular embodiments, the oligonucleotide is as described herein for oligonucleotides and/or the polymer is as described herein or otherwise known in the art or subsequently identified.

In yet another aspect, the invention provides an oligonucleotide randomer, where the randomer is at least 6 nucleotides in length. In particular embodiments the randomer has a length as specified above for oligonucleotides; the randomer includes at least one phosphorothioate linkage, the randomer includes at least one phosphorodithioate linkage or other modification as listed herein; the randomer oligonucleotides include at least one non-randomer segment (such as a segment complementary to a selected nucleic acid sequence), which can have a length as specified above for oligonucleotides; the randomer is in a preparation or pool of preparations containing at least 5, 10, 15, 20, 50, 100, 200, 500, or 700 micromol, 1, 5, 7, 10, 20, 50, 100, 200, 500, or 700 mmol, or 1 mole of randomer, or a range defined by taking any two different values from the preceding as inclusive end points, or is synthesized at one of the listed scales or scale ranges.

In connection with modifying characteristics of an oligonucleotide by linking or conjugating with another molecule or moiety, the modifications in the (characteristics are evaluated relative to the same oligonucleotide without the linked or conjugated molecule or moiety.

### Phosphorothioation and 2′ Sugar Modification

The incorporation of phosphorothioate linkages and ribonucleotide modifications, including 2′-O-methyl and other 2′ sugar modifications, into oligonucleotides of this invention, may be useful for improving characteristics of sequence-independent therapeutic oligonucleotides. Results demonstrate that modification at the 2′-position of each ribose reduces the general interaction of the PS-ONs with serum proteins and renders them significantly more resistant to low pH. These properties promise to increase the pharmacokinetic performance and reduce the toxic side effects normally seen with PS-ONs. For example, their pH resistance makes them more suitable for oral delivery. Also their lowered interaction with serum proteins promises to improve their pharmacokinetic behaviour without affecting their therapeutic activity. Thus, oligonucleotides having each linkage phosphorothioated and each ribonucleotide modified at the 2′-position of the ribose, e.g., 2′-O-methyl modifications, may have therapeutic activity but do not trigger RNase H activity, a property desirable for traditional antisense ONs but completely dispensable for the activity described in this present invention. Results also demonstrate that modifications at the 2′-position of each ribose of PS-ONs renders the ON more resistant to nucleases in comparison with a PS-ON comprising the same modifications but only at both ends (gapmer). Gapmers are preferentially used in the antisense technology. Nuclease resistance of PS-ONs including modifications at the 2′-position of each ribose could display beneficial properties, such as improved pharmakokinetics and/or oral availability.

In addition, while PS-ONs that include modifications at the 2′-position of each ribose show desirable characteristics, PS-ONs with substantial numbers of modifications at the 2′-position of ribose could also display desirable characteristics, e.g., modification to at least 50% of the riboses and more preferably 80% or even more.

### Oligonucleotide Modifications and Synthesis

As indicated herein, modified ONs may be useful in this invention. Such modified ONs include, for example, ONs containing modified backbones or non-natural internucleoside linkages. ONs having modified backbones include those that retain a phosphorus atom in the backbone and those that do not have a phosphorus atom in the backbone.

Such modified ON backbones include, for example, phosphorothioates, chiral phosphorothioates, phosphorodithioates, phosphotriesters aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates, carboranyl phosphate and borano-phosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity typically include a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Preparation of oligonucleotides with phosphorus-containing linkages as indicated above are described, for example, in U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, each of which is incorporated by reference herein in its entirety.

Some exemplary modified ON backbones that do not include a phosphodiester linkage have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, 0, S and CH₂ component parts. Particularly advantageous are backbone linkages that include one or more charged moieties. Examples of U.S. patents describing the preparation of the preceding oligonucleotides include U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is incorporated by reference herein in its entirety.

Modified ONs may also contain one or more substituted sugar moieties. For example, such oligonucleotides can include one of the following 2′-modifications: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl, or 2′-O—(O-carboran-1-yl)methyl. Particular examples are O[(CH₂)ₙO]ₘCH₃, O(CH₂)˜OCH₃, O(CH₂)ₙNH₂, O(CH₂)ₙCH₃, O(CH₂)ₙONH₂, and O(CH₂)ₙON [(CH₂)ₙCH₃)]₂, where n and m are from 1 to 10. Other exemplary ONs include one of the following 2′-modifications: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃. OCF₃, SOCH₃, SO₂CH₃, ONO₂, NO₂, N₃, NH₂, heterocycloalkyl, heterocycloalkaryl, aminoalkylamino, polyalkylamino, substituted silyl, a reporter group, an intercalator, a group for improving the pharmacokinetic properties of an ON, or a group for improving the pharmacodynamic properties of an ON. Examples include 2′-methoxyethoxy(2′-O—CH₂CH₂OCH₃, also known as 2′-O-(2-methoxyethyl) or 2′-MOE) (Martin et al., 1995, Helv. Chim. Acta, 78: 486-504) i.e., an alkoxyalkoxy group; 2′-dimethyl-laminooxyethoxy, i.e., a O(CH₂)₂ON(CH₃)₂ group, also known as 2′-DMAOE; and 2′-(dimethylaminoethoxyethoxy (also known as 2′-O— dimethylaminoethoxyethyl or 2′-DMAEOE), i.e., 2′-O—CH₂—O—CH₂—N(CH₂)₂.

Other modifications include Locked Nucleic Acids (LNAs) in which the 2′-hydroxyl group is linked to the 3′ or 4′ carbon atom of the sugar ring thereby forming a bicyclic sugar moiety. The linkage can be a methelyne (—CH₂—)˜ group bridging the 2′ oxygen atom and the 4′ carbon atom wherein n is 1 or 2. LNAs and preparation thereof are described in International patent application publication Nos WO 98/39352 and WO 99/14226, which are incorporated herein by reference in their entireties.

Other modifications include sulfur-nitrogen bridge modifications, such as locked nucleic acid as described in Orum et al. (2001, Curr. Opin. Mol. Ther. 3: 239-243).

Other modifications include 2′-methoxy(2′-O—CH₃), 2′-methoxyethyl (2′O—CH₂—CH₃), 2′-ethyl, 2′-ethoxy, 2′-aminopropoxy(2′-OCH₂CH₂CH₂NH₂), 2′-allyl (2′-CH₂—CH═CH₂), 2′-O-allyl (2′-O—CH₂—CH═CH₂) and 2′-fluoro (2′-F).

The 2′-modification may be in the arabino (up) position or ribo (down) position. Similar modifications may also be made at other positions on the ON, particularly the 3′ position of the sugar on the 3′ terminal nucleotide or in 2′-5′ linked oligonucleotides and the 5′ position of the 5′ terminal nucleotide. ONs may also have sugar mimetics such as cyclobutyl moieties in place of the pentofuranosyl sugar. Exemplary U.S. patents describing the preparation of such modified sugar structures include, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is incorporated by reference herein in its entirety.

Still other modifications include an ON concatemer consisting of multiple ON sequences joined by a linker(s). The linker may, for example, consist of modified nucleotides or non-nucleotide units. In some embodiments, the linker provides Flexibility to the ON concatemer. Use of such ON concatemers can provide a facile method to synthesize a final molecule, by joining smaller ON building blocks to obtain the desired length. For example, a 12 carbon linker (C12 phosphoramidite) can be used to join two or more ON concatemers and provide length, stability, and flexibility.

As used herein, “unmodified” or “natural” bases (nucleobases) include the purine bases adenine (A) and guanine (G), and the pyrimidine bases thymine (T), cytosine (C) and uracil (U). ONs may also include base modifications or substitutions. Modified bases include other synthetic and naturally-occurring bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl(—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Additional modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Further nucleobases include those described in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993.

Another modification includes phosphorodithioate linkages. Knowing that phosphorodithioate ONs (PS2-ONs) and PS-ONs have both binding affinity to proteins (Tonkinson et al., 1994, Antisense Res. Dev. 4: 269-278; Cheng et al., 1997, J. Mol. Recogn. 10: 101-107) and knowing that a possible mechanism of action of ONs is binding to protein involved in diseases, it could be desirable to include phosphorodithioate linkages on the therapeutic ONs described in this invention.

Another approach to modify ONs is to produce stereodefined or stereo-enriched ONs as described in Yu et al. (2000, Bioorg. Med. Chem. 8: 275-284) and in Inagawa et al. (2002, FEBS Lett. 25: 48-52). ONs prepared by conventional methods consist of a mixture of diastereomers by virtue of the asymmetry around the phosphorus atom involved in the internucleotide linkage. This may affect the stability of the binding between ONs and targets such as proteins involved in diseases. Previous data showed that protein binding is significantly stereo-dependent (Yu et al., 2000, Bioorg. Med. Chem. 8: 275-284). Thus, using stereodefined or stereo-enriched ONs could improve their protein binding properties and improve their therapeutic efficacy.

The incorporation of modifications such as those described above can be utilized in many different incorporation patterns and levels. That is, a particular modification need not be included at each nucleotide or linkage in an ON, and different modifications can be utilized in combination in a single ON, or even on a single nucleotide.

As examples and in accordance with the description above, modified oligonucleotides containing phosphorothioate or dithioate linkages may also contain one or more substituted sugar moieties particularly modifications at the sugar moieties including, without restriction, 2′-ethyl, 2′-ethoxy, 2′-methoxy, 2′-aminopropoxy, 2′-allyl, 2′-fluoro, 2′-pentyl, 2′-propyl, 2′-dimethylaminooxyethoxy, and 2′-dimethylaminoethoxyethoxy. The 2′-modification may be in the arabino (up) position or ribo (down) position. A preferred 2′-arabino modification is 2′-fluoro. Similar modifications may also be made at other positions on the ON, particularly the 3′ position of the sugar on the 3′ terminal nucleotide or in 2′-5′ linked oligonucleotides and the 5′ position of 5′ terminal nucleotide. ONs may also have sugar mimetics such as cyclobutyl moieties in place of the pentofuranosyl sugar. Moreover ONs may have a structure of or comprise a portion consisting of glycol nucleic acid (GNA) with an acyclic propylene glycol phosphodiester backbone (Zhang et al., 2005, J. Am. Chem. Soc. 127(12): 4174-4175). Such GNA may comprise phosphorothioate linkages and may comprise only pyrimidine bases.

### Oligonucleotide Formulations and Pharmaceutical Compositions

The oligonucleotides of the present invention can be prepared in an ON formulation or pharmaceutical composition. Thus, the present ONs may also be mixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Exemplary United States patents that describe the preparation of such uptake, distribution and/or absorption assisting formulations include, for example, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is incorporated herein by reference in its entirety.

The ONs, formulations, and compositions of the invention include any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular embodiments, prodrug versions of the present oligonucleotides are prepared as SATE [(S-acetyl-2-thioethyl)phosphate] derivatives according to the methods disclosed in Gosselin et al. (International patent application publication No WO 93/24510 and in Imbach et al. (International patent application publication No WO 94/26764 and U.S. Pat. No. 5,770,713), which are hereby incorporated by reference in their entireties.

The term “pharmaceutically acceptable salts” refers to physiologically and pharmaceutically acceptable salts of the present compounds: i.e., salts that retain the desired biological activity of the parent compound and do not impart undesired toxicological effects thereto. Many such pharmaceutically acceptable salts are known and can be used in the present invention.

For ONs, useful examples of pharmaceutically acceptable salts include but are not limited to salts formed with cations such as sodium, potassium, ammonium, magnesium, calcium, polyamines such as spermine and spermidine, etc.; acid addition salts formed with inorganic acids, for example hydrochloric acid, hydrobromic acid, sulfuric acid, phosphoric acid, nitric acid and the like; salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and salts formed from elemental anions such as chlorine, bromine, and iodine.

The present invention also includes pharmaceutical compositions and formulations which contain the therapeutic ONs of the present invention.

Examples of administrations include topical (including ophthalmic and to mucous membranes including vaginal and rectal delivery or by enema); pulmonary, e.g., by inhalation or insufflations of powders or aerosols, including by nebulizer; intratracheal; intracerebral; by intracerebral implant, intranasal; epidermal and transdermal; oral; or parenteral. Parenteral administration includes intravenous, intraarterial, subcutaneous, intraperitoneal or intramuscular injection or infusion.

Pharmaceutical compositions and formulations for administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Other formulations include those in which the ONs of the invention are mixed with a topical delivery agent such as lipids, liposomes, fatty acids, fatty acid esters, steroids, chelating agents and surfactants. Preferred lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP, dioleoylphosphatidyl ethanolamine DOTMA) and other delivering agents or molecules. ONs may be encapsulated within liposomes or may form complexes thereto, in particular to cationic liposomes. Alternatively, ONs may be complexed to lipids, in particular to cationic lipids. Preferred fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g. isopropylmyristate IPM), monoglyceride, diglyceride or pharmaceutically acceptable salt thereof.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Preferred oral formulations are those in which oligonucleotides of the invention are administered in conjunction with one or more penetration enhancers, surfactants and chelators. Exemplary surfactants include fatty acids and/or esters or salts thereof, bile acids and/or salts thereof. Exemplary bile acids/salts include chenodeoxycholic acid (CDCA) and ursodeoxychenedeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate, sodium glycodihydrofusidate. Exemplary fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g. sodium). Also preferred are combinations of penetration enhancers, for example, fatty acids/salts in combination with bile acids/salts. A particularly preferred combination is the sodium salt of lauric acid, capric acid and UDCA. Further exemplary penetration enhancers include polyoxyethylene-9-lauryl ether, polyoxyethylene-20-cetyl ether. Oligonucleotides of the invention may be delivered orally in granular form including sprayed dried particles, or complexed to form micro or nanoparticles. ON complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses, and starches. Particularly advantageous complexing agents include chitosan, N-trimethylchitosan, poly-L-lysine, polyhistidine, polyorithine, polyspermines, protamine, polyvinylpyridine, polythiodiethylamino-methylethylene P(TDAE), polyaminostyrene (e.g. p-amino), poly(methylcyanoacrylate), poly(ethylcyanoacrylate), poly(butylcyanoacrylatc), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG).

Compositions and formulations for parenteral administration may include sterile aqueous solutions which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaking the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention.

### Emulsions

The formulations and compositions of the present invention may be prepared and formulated as emulsions. Emulsions are typically heterogeneous systems of one liquid dispersed in another in the form of droplets usually exceeding 0.1 μm in diameter. (Idson, in Pharmaceutical Dosage Forms, Lieberman, Rieger and Banker (lids.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et at., in Remington's Pharmaceutical Sciences, Mack Publishing Co., Easton, Pa., 1985, p. 301). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: non-ionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

Large varieties of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong inter-facial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The applications of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of reasons of ease of formulation, efficacy from an absorption and bioavailability standpoint. (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of ONs are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically micro-emulsions are systems that are prepared by first dispersing oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML31O), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschet, Meth. Find. Exp. Clin. Pharmacol, 1993, 13, 205). Micro-emulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Set, 1996, 85, 138-143). Often microemulsions may form spontaneously when their components are brought together at ambient temperature. This may be particularly advantageous when formulating thermolabile drugs, peptides or oligonucleotides. Microemulsions have also been effective in the transdermal delivery of active components in both cosmetic and pharmaceutical applications. It is expected that the microemulsion compositions and formulations of the present invention will facilitate the increased systemic absorption of ONs and nucleic acids from the gastrointestinal tract.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92).

### Liposomes

There are many organized surfactant structures besides microemulsions that have been studied and used for the formulation of drugs. These include monolayers, micelles, bilayers and vesicles. Vesicles offer specificity and extended duration of action for drug delivery. Thus, as used herein, the term “liposome” refers to a vesicle composed of amphiphilic lipids arranged in a spherical bilayer or bilayers, i.e., liposomes are unilamellar or multilamellar vesicles which have a membrane formed from a lipophilic material and an aqueous interior. The aqueous portion typically contains the composition to be delivered. In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores. Additional factors for liposomes include the lipid surface charge, and the aqueous volume of the liposomes.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245).

For topical administration, there is evidence that liposomes present several advantages over other formulations. Such advantages include reduced side-effects related to high systemic absorption of the administered drug, increased accumulation of the administered drug at the desired target.

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome include one or more glycolipids, such as monosialoganglioside G_{M1}, or is derivatized with one or more hydrophilic polymers, such as a polyethylene glycol (PEG) moiety. Without being bound by any particular theory, it is believed that for sterically stabilized liposomes containing gangliosides, sphingomyelin, or PEG-derivatized lipids, the increase in circulation half-life of these sterically stabilized liposomes is due to a reduced uptake into cells of the reticuloendothelial system (RES) (Allen et al., FEBS Lett., 1987, 223, 42; Wu et al., Cancer Research, 1993, 53, 3765).

Various liposomes that include one or more glycolipids have been reported in Papahadjopoulos et al., Ann. N.Y. Acad. Sci., 1987, 507, 64 (monosiatoganglioside G_{M1}, galactocerebroside sulfate and phosphatidylinositol); Gabizon et al., Proc. Natl. Acad. Sci. USA., 1988, 85, 6949; Allen et al., U.S. Pat. No. 4,837,028 and International application publication No WO 88/04924 (sphingomyelin and the ganglioside G_{M1} or a galactocerebroside sulfate ester); Webb et al., U.S. Pat. No. 5,543,152 (sphingomyelin); Lim et al., International patent application publication No WO 97/13499 (1,2-sn-dimyristoylphosphatidylcholine).

Liposomes that include lipids derivatized with one or more hydrophilic polymers, and methods of preparation are described, for example, in Sunamoto et al., Bull. Chem. Soc. Jpn., 1980, 53, 2778 (a nonionic detergent, 2C₁₂15G, that contains a PEG moiety); Illum et al., FEBS Lett., 1984, 167, 79 (hydrophilic coating of polystyrene particles with polymeric glycols); Sears, U.S. Pat. Nos. 4,426,330 and 4,534,899 (synthetic phospholipids modified by the attachment of carboxylic groups of polyalkylene glycols (e.g., PEG)); Klibanov et al., FEBS Lett., 1990, 268, 235 (phosphatidylethanolamine (PE) derivatized with PEG or PEG stearate); Blume et al., Biochimica et Biophysica Acta, 1990, 1029, 91 (PEG-derivatized phospholipids, e.g., DSPE-PEG, formed from the combination of distearoylphosphatidylethanolamine (DSPE) and PEG); Fisher, European Patent No. EP 0 445 131 B1 and International patent application publication No WO 90/04384 (covalently bound PEG moieties on liposome external surface); Woodle et al., U.S. Pat. Nos. 5,013,556 and 5,356,633, and Martin et al., U.S. Pat. No. 5,213,804 and European Patent No EP 0 496 813 B1 (liposome compositions containing 1-20 mole percent of PE derivatized with PEG); Martin et al., International patent application publication No WO 91/05545 and U.S. Pat. No. 5,225,212 and in Zalipsky et al., International patent application publication No WO 94/20073 (liposomes containing a number of other lipid-polymer conjugates); Choi et al., International patent application publication No WO 96/10391 (liposomes that include PEG-modified ceramide lipids); Miyazaki et al., U.S. Pat. No. 5,540,935, and Tagawa et al., U.S. Pat. No. 5,556,948 (PEG-containing liposomes that can be further derivatized with functional moieties on their surfaces).

Liposomes that include nucleic acids have been described, for example, in Thierry et al., International patent application publication No WO 96/40062 (methods for encapsulating high molecular weight nucleic acids in liposomes); Tagawa et al., U.S. Pat. No. 5,264,221 (protein-bonded liposomes containing RNA); Rahman et al., U.S. Pat. No. 5,665,710 (methods of encapsulating oligodeoxynucleotides in liposomes); Love et al., International patent application publication No WO 97/04787 (liposomes that include antisense oligonucleotides).

Another type of liposome, transfersomes are highly deformable lipid aggregates which are attractive for drug delivery vehicles. (Cevc et al., 1998, Biochim Biophys Acta. 1368(2): 201-215.) Transfersomes may be described as lipid droplets which are so highly deformable that they can penetrate through pores which are smaller than the droplet. Transfersomes are adaptable to the environment in which they are used, for example, they are shape adaptive, self-repairing, frequently reach their targets without fragmenting, and often self-loading. Transfersomes can be made, for example, by adding surface edge-activators, usually surfactants, to a standard liposomal composition.

### Surfactants

Surfactants are widely used in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants are widely used in pharmaceutical and cosmetic products and are usable over a wide range of pH values, and with typical HLB values from 2 to about 18 depending on structure. Nonionic surfactants include nonionic esters such as ethylene glycol esters, propylene glycol esters, glyceryl esters, polyglyceryl esters, sorbitan esters, sucrose esters, and ethoxylated esters; and nonionic alkanolamides and ethers such as fatty alcohol ethoxylates, propoxylated alcohols, and ethoxylated/propoxylated block polymers are also included in this class. The polyoxyethylene surfactants are the most commonly used members of the nonionic surfactant class.

Surfactant molecules that carry a negative charge when dissolved or dispersed in water are classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isothionates, acyl laurates and sulfosuccinates, and phosphates. The alkyl sulfates and soaps are the most commonly used anionic surfactants.

Surfactant molecules that carry a positive charge when dissolved or dispersed in water are classified as cationic. Cationic surfactants include quaternary ammonium salts and ethoxylated amines, with the quaternary ammonium salts used most often.

Surfactant molecules that can carry either a positive or negative charge are classified as amphoteric. Amphoteric surfactants include acrylic acid derivatives, substituted alkylamides, N-alkylbetaines and phosphatides.

The use of surfactants in drug products, formulations and in emulsions has been reviewed in Rieger, in Pharmaceutical Dosage Forms, Marcel Dekker, Inc., New York, N.Y., 1988, p. 285).

### Penetration Enhancers

In some embodiments, penetration enhancers are used in or with a composition to increase the delivery of nucleic acids, particularly ONs across membranes of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating nonsurfactants (Lee et al., Critical Reviews in Therapeutic Drug Carrier Systems, 1991, p. 92). Each of these classes of penetration enhancers is described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of ONs through the mucosa is enhanced. These penetration enhancers include, for example, sodium lauryl sulfate, polyoxyethylene-9-lauryl ether and polyoxyethylene-20-cetyl ether) (Lee et al., Critical Reviews in Therapeutic Drug Carrier Systems, 1991, p. 92); and perfluorochemical emulsions, such as FC-43. Takahashi et al., J. Pharm. Pharmacol., 1988, 40, 252), each of which is incorporated herein by reference in its entirety.

Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₁₀ alkyl esters thereof (e.g., methyl, isopropyl and t-butyl), and mono- and diglycerides thereof (i.e., oleate, laurate, caprate, myristate, palmitate, stearate, linoleate, etc.) (Lee et al., Critical Reviews in Therapeutic Drug Carrier Systems, 1991, p. 92; Muranishi, Critical Reviews in Therapeutic Drug Carrier Systems, 1990, 7, 1-33; El Hariri et al., J. Pharm. Pharmacol., 1992, 44, 651-654), each of which is incorporated herein by reference in its entirety.

The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al., Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263: 25; Yamashita et al., J. Pharm.: Sci., 1990, 79: 579-583).

In the present context, chelating agents can be regarded as compounds that remove metallic ions from solution by forming complexes therewith, with the result that absorption of ONs through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618: 315-339). Without limitation, chelating agents include disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14: 43-51).

As used herein, non-chelating non-surfactant penetration enhancing compounds are compounds that do not demonstrate significant chelating agent or surfactant activity, but still enhance absorption of oligonucleotides through the alimentary mucosa (Muranishi, Critical Reviews in Therapeutic Drug Carrier Systems, 1990, 7: 1-33). Examples of such penetration enhancers include unsaturated cyclic ureas, 1-alkyl- and 1-alkenylazacyclo-alkanone derivatives (Lee et al., Critical Reviews in Therapeutic Drug Carrier Systems, 1991, page 92); and nonsteroidal therapeutic agents such as diclofenac sodium, indomethacin and phenylbutazone (Yamashita et al., J. Pharm. Pharmacol., 1987, 39, 621-626).

Other agents may be utilized to enhance the penetration of the administered nucleic acids, including glycols such as ethylene glycol and propylene glycol, pyrrols such as 2-pyrrol, azones, and terpenes such as limonene and menthone.

### Carriers

Certain compositions of the present invention also incorporate carrier compounds in the formulation. As used herein, “carrier compound” or “carrier” can refer to a nucleic acid, or an analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, often with an excess of the latter substance, can result in a substantial reduction of the amount of nucleic acid recovered in the liver, kidney or other extracirculatory reservoirs. For example, the recovery of a partially phosphorothioated ON in hepatic tissue can be reduced when it is coadministered with polyinosinic acid, dextran sulfate, polycytidic acid or 4-acetamido-4′isothiocyano-stilbene-2,2-disulfonic acid (Miyao et al., Antisense Res. Dev., 1995, 5: 115-121; Takakura et al., Antisense & Nucl Acid Drug Dev., 1996, 6: 177-183), each of which is incorporated herein by reference in its entirety.

### Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal, and is typically liquid or solid. A pharmaceutical carrier is generally selected to provide for the desired bulk, consistency, etc., when combined with a nucleic acid and the other components of a given pharmaceutical composition, in view of the intended administration mode. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycotate, etc.); and wetting agents (e.g., sodium lauryl sulphate, etc.).

Pharmaceutically acceptable organic or inorganic excipients suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

### Other Pharmaceutical Composition Components

The present compositions may additionally contain other components conventionally found in pharmaceutical compositions, at their art-established usage levels. Thus, for example, the compositions may contain additional, compatible, pharmaceutically-active materials such as, antipruritics, astringents, local anesthetics or therapeutic agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran, and/or stabilizers.

Certain embodiments of the invention provide pharmaceutical compositions containing (a) one or more therapeutic ONs and (b) one or more other agents used which function by similar or different mechanisms and targeting the same disease. Examples of such agent include for thrombotic disorders, without limitation, antiplatelet agents (including aspirin and other non-steroidal anti-inflammatory agents such as ibuprofen), anticoagulant agents (including heparin ant its derivates), vitamin K antagonists (including coumarin derivatives and warfarin) and thrombolytic agents (including tPA, streptokinase, urokinase prourokinase, anisolylated plasminogen streptokinase activation complex (APSAC), and animal salivary gland plasminogen activators). Examples of such agent include for cholesterol related disorders, without limitation, bile acid sequestrants, nicotinic acid (niacin), 3-hydroxy-3-methylglutaryl coenzyme A (HMG CoA) reductase inhibitors, probucol (Lorelco™), fibrate derivatives (bezafibrate, fenofibrate, gemfibrozil, clofibrate, Lovastatin, Simvastatin, Pravastatin and Fluvastatin), Neomycin and cholestyramine. Examples of such agent include for osteroporosis, without limitation, anti-resorptive compounds to reduce the resorption of bone tissue (including oestrogen) and anabolic agents to promote bone formation and increase bone mass. Examples of such agent include for snake venom effects, without limitation, antivenins.

Administration of ONs of this invention used in the pharmaceutical composition or formulation or to practice a method of treating a human or an animal can be carried out in a variety of conventional ways for example using ocular, oral, subcutaneous, intravenous, intraperitoneal, intramuscular, intrathecal, intracerebral, by intracerebral implant, intranasal, by inhalation, by enema, transdermal, sublingual, intra arterial, intra tracheal intra urethral and dermal routes.

The pharmaceutical composition or ON formulation of the invention may further contain other drugs for the treatment of diseases. Such additional factors and/or agents may be included in the pharmaceutical composition, for example, to produce a synergistic effect with the ONs of the invention.

In another approach, therapeutic ONs demonstrating low, preferably the lowest possible, homology with the human (or other subject organism's) genome is designed. The oligonucleotide has at most 60%, preferably 50%, more preferably 40% identity with a genomic sequence. One goal is to obtain an ON that will show the lowest toxicity due to interactions with human or animal genome sequence(s) and/or mRNAs. The first step is to produce the desired length sequence of the ON, e.g., by aligning nucleotides A, C, G, T/U in a random fashion, manually or, more commonly, using a computer program. The second step is to compare the ON sequence with a library of human sequences such as GenBank and/or the Ensemble Human Genome Database. The sequence generation and comparison can be performed repetitively, if desired, to identify a sequence or sequences having a desired low homology level with the subject genome. It is desirable for the ON sequence to have the lowest homology possible with the entire genome, while also minimizing self interaction. The last step is to test the ON in an assay to measure therapeutic activity.

In another approach, sequence independent ON sequence portion(s) is/are coupled with antisense sequence portion(s) to increase the activity of the final ON. The non-specific portion of the ON is described in the present invention. The antisense portion can be complementary to an inflammatory gene mRNA or to other genes important for the progression of diseases.

In another approach, sequence independent sequence portion(s) is/are coupled with a G-rich motif ON portion(s) to improve the activity of the final ON. The non-specific portion of the ON is described in the present invention. The G-rich motif portion can, as non-limiting examples, include, CpG, Gquartet, and/or CG that are described in the literature as stimulators of the immune system.

Another approach is to use an ON composed of one or more types of non-Watson-Crick nucleotides/nucleosides. Such ONs can mimic PS-ONs and other modifications with some of the following characteristics similar to PS-ONs: a) the total charge; b) the space between the units; c) the length of the chain; d) a net dipole with accumulation of negative charge on one side; e) the ability to bind to proteins f) the ability to be used with delivery systems, h) an acceptable therapeutic index, i) a therapeutic activity. The ON can have a phosphorothioate backbone but is not limited to it. Examples of non-Watson-Crick nucleotides/nucleosides are described in Kool, 2002, Acc. Chem. Res. 35:936-943; and Takeshita et al., (1987) J. Biol. Chem. 262: 10171-10179 where ONs containing synthetic abasic sites are described.

Another approach is to use a polymer mimicking the activity of ONs described in the present invention to obtain inhibition of diseases activity. As described in the literature, several anionic polymers were shown to bind to proteins. These polymers belong to several classes: (1) sulfate esters of polysaccharides (dextrin and dextran sulfates; cellulose sulfate); (2) polymers containing sulfonated benzene or naphthalene rings and naphthalene sulfonate polymers; (3) polycarboxylates (acrylic acid polymers); and acetyl phthaloyl cellulose (Neurath et al., 2002, BMC Infect Dis 2:27); and (4) abasic ONs (Takeshita et al., 1987, J. Biol. Chem. 262: 10171-10179). Other examples of non-nucleotide protein binding polymers are described in the literature. The polymers described herein can mimic ONs described in this invention and may have some or all of the following characteristics similar to ONs: a) the length of the chain; b) a net dipole with accumulation of negative charge on one side; c) the ability to bind to proteins; d) the ability to be encapsulated by a delivery system, e) an acceptable therapeutic index, f) a therapeutic activity. In order to mimic the effect of an ON, the therapeutic polymer may preferably be a polyanion displaying similar space between its units as compared to a PS-ON for example, without limitation, using a 3 carbon linker in place of a standard nucleotide. Also to mimic the effect of an ON, the therapeutic polymer may display a similar hydrophobicity than PS-ON.

The present invention would be readily understood by referring to the following examples which are given to illustrate the invention rather than limits its scope.

ONs that may be used in this invention are listed in the following:

| | | | |
|---|---|---|---|
| ON | SEQUENCE | SIZE | MODIFICATION(s) |

| | | | |
|---|---|---|---|
| REP 2001 | GAA GCG TTC GCA CTT CGT CCC A | 22 | PS |
| | (SEQ ID NO: 1) | | |
| REP 2002 | NNNNN | 5 | PS |
| | (SEQ ID NO: 2) | | |
| REP 2003 | NNNNNNNNNN | 10 | PS |
| | (SEQ ID NO: 3) | | |
| REP 2004 | NNNNNNNNNNNNNNNNNNNN | 20 | PS |
| | (SEQ ID NO: 4) | | |
| REP 2005 | NNNNNNNNNNNNNNNNNNNNNNNNNNNNNN | 30 | PS |
| | (SEQ ID NO: 5) | | |
| REP 2006 | NNNNNNNNNNNNNNNNNNNNNNNNNNNNNNNNNNNNNNNN | 40 | PS |
| | (SEQ ID NO: 6) | | |
| REP 2007 | NNNNNNNNNNNNNNNNNNNNNNNNNNNNNNNNNNNNNNNNNNNN | 80 | PS |
| | NNNNNNNNNNNNNNNNNNNNNNNNNNNNNNNNNNNN | | |
| | (SEQ ID NO: 7) | | |
| REP 2008 | NNNNNNNNNNNNNNNNNNNNNNNNNNNNNNNNNNNNNNNNNNNN | 120 | PS |
| | NNNNNNNNNNNNNNNNNNNNNNNNNNNNNNNNNNNNNNNNNNNN | | |
| | NNNNNNNNNNNNNNNNNNNNNNNNNNNNNNNN | | |
| | (SEQ ID NO: 8) | | |
| REP 2009 | NNNNNNNNNNNN | 12 | PS |
| | (SEQ ID NO: 9) | | |
| REP 2010 | NNNNNNNNNNNNNN | 14 | PS |
| | SEQ ID NO: 10) | | |
| REP 2011 | NNNNNNNNNNNNNNNN | 16 | PS |
| | (SEQ ID NO: 11) | | |
| REP 2012 | NNNNNNNNNNNNNNNNNN | 18 | PS |
| | (SEQ ID NO: 12) | | |
| REP 2013 | NNNNNNNNNN | 10 | unmodified |
| | (SEQ ID NO: 13) | | |
| REP 2014 | NNNNNNNNNNNNNNNNNNNN | 20 | unmodified |
| | (SEQ ID NO: 14) | | |
| REP 2015 | NNNNNNNNNNNNNNNNNNNNNNNNNNNNNNNNNNNNNNNN | 40 | unmodified |
| | (SEQ ID NO: 15) | | |
| REP 2016 | tccgaagacg | 10 | PS |
| | (SEQ ID NO: 16) | | |
| REP 2017 | acacctccgaagacgataac | 20 | PS |
| | (SEQ ID NO: 17) | | |
| REP 2018 | ctacagacatacacctccgaagacgataacactagacata | 40 | PS |
| | (SEQ ID NO: 18) | | |
| REP 2019 | cccccatgga | 10 | PS |
| | (SEQ ID NO: 19) | | |
| REP 2020 | tacgacccccatggagcccc | 20 | PS |
| | (SEQ ID NO: 20) | | |
| REP 2021 | tccagccgcatacgacccccatggagccccgccccggagc | 40 | PS |
| | (SEQ ID NO: 21) | | |
| REP 2022 | NNNNNNNNNNNNNNNNNNNNNNNNNNNNNNNNNNNNNN | 40 | full 2-O-Me(N) |
| | NN | | |
| | (SEQ ID NO: 22) | | |
| REP 2023 | NNNNNNNNNNNNNNNNNNNNNNNNNNNNNNNNNNNNNNNN | 40 | 2-O-Me(N)/ |
| | (SEQ ID NO: 23) | | unmodified |
| REP 2024 | NNNNNNNNNNNNNNNNNNNNNNNNNNNNNNNNNNNNNNNN | 40 | 2-O-Me(N)/PS |
| | (SEQ ID NO: 24) | | |
| REP 2025 | AGAGNNNNNNNNNNNNNNNNNNNNNNNNNNNNNNNNNNNNAGA | 40 | LNA/unmodified |
| | G | | |
| | (SEQ ID NO: 25) | | |
| REP 2026 | NNNNNNNNNNNNNNNNNNNNNNNNNNNNNNNNNNNNNNNN | 40 | MePhos(N)/ |
| | (SEQ ID NO: 26) | | unmodified |
| REP 2027 | NNNNNNNNNNNNNNNNNNNNNNNNNNNNNNNNNNNNNNNN | 40 | MePhos(N)/ |
| | (SEQ ID NO: 27) | | unmodified |
| REP 2028 | GGGGGGGGGGGGGGGGGGGGGGGGGGGGGGGGGGGGGGGG | 40 | PS |
| | (SEQ ID NO: 28) | | |
| REP 2029 | AAAAAAAAAAAAAAAAAAAAAAAAAAAAAAAAAAAAAAAA | 40 | PS |
| | (SEQ ID NO: 29) | | |
| REP 2030 | TTTTTTTTTTTTTTTTTTTTTTTTTTTTTTTTTTTTTTTT | 40 | PS |
| | (SEQ ID NO: 30) | | |
| REP 2031 | CCCCCCCCCCCCCCCCCCCCCCCCCCCCCCCCCCCCCCCC | 40 | PS |
| | (SEQ ID NO: 31) | | |
| REP 2032 | NNNNNN | 6 | PS |
| | (SEQ ID NO: 32) | | |
| REP 2033 | TGTGTGTGTGTGTGTGTGTGTGTGTGTGTGTGTGTGTGTG | 40 | PS |
| | (SEQ ID NO: 33) | | |
| REP 2034 | TGTGTGTGTGTGTGTGTGTG | 20 | PS |
| | (SEQ ID NO: 34) | | |
| REP 2035 | TGTGTGTGTG | 10 | PS |
| | (SEQ ID NO: 35) | | |
| REP 2036 | GCGTTTGCTCUCUCTTGCG | 21 | PS |
| | (SEQ ID NO: 36) | | |
| REP 2037 | CTCTCGCACCCATCTCTCTCCTTCT | 25 | PS |
| | (SEQ ID NO: 37) | | |
| REP 2038 | UCGCACCCATCTCTCTCCUUC | 21 | 2-O-Me(N)/PS |
| | (SEQ ID NO: 38) | | |
| REP 2043 | NNNNNNNNNNNNNNNNNNNNNNNNNNNNNNNNNNNNNNNNNNN | 48 | 2-O-Me(N)/PS |
| | NNNNNNN | | |
| | (SEQ ID NO. 39) | | |
| REP 2044 | NNNNNNNNNNNNNNNNNNNNNNNNNNNNNNNNNNNNNNNNNNN | 68 | 2-O-Me(N)/PS |
| | NNNNNNNNNNNNNNNNN | | |
| | (SEQ ID NO: 40) | | |
| REP 2045 | NNNNNNNNNNNNNNNNNNNNNNNNNNNNNNNNNNNNNNNNNNNN | 60 | PS |
| | NNNNNNNNNNNNNNNN | | |
| | (SEQ ID NO: 41) | | |
| REP 2046 | CCCCCGGGGGGTGTGTTTCGGGGGGGGCCC | 30 | PS |
| | (SEQ ID NO: 42) | | |
| REP 2047 | CCGCGCGCGACCCCCGGGGGGTGTGTTTCGGGGGGGGCCC | 40 | PS |
| | (SEQ ID NO: 43) | | |
| REP 2048 | CGACTGGTGAGTACGCCAAAAATTTTGACTAGCGGAGGCT | 40 | PS |
| | (SEQ ID NO: 44) | | |
| REP 2049 | AGUAGAAACAAGGGUG-linker-CACCCUGCUUUUGCU | 32 | PS RNA |
| | (SEQ ID NO: 45) (SEQ ID NO: 46) | | |
| REP 2050 | AGTAGAAACAAGGGTG-linker-CACCCTGCTTTTGCT | 32 | PS |
| | (SEQ ID NO: 47) (SEQ ID NO: 48) | | |
| REP 2051 | AGUAGAAACAAGGGUGNNNNNNNNNNNNNNNNNNNNNNNNNNN | 71 | PS RNA and DNA |
| | NNNNNNNNNNNNNCACCCUGCUUUUGCU | | |
| | (SEQ ID NO: 49) | | |
| REP 2052 | AGTAGAAACAAGGGTCNNNNNNNNNNNNNNNNNNNNNNNNNNNN | 71 | PS |
| | NNNNNNNNNNNNCACCCTGCTTTTGCT | | |
| | (SEQ ID NO: 50) | | |
| REP 2055 | ACACACACACACACACACACACACACACACACACACACAC | 40 | PS |
| | (SEQ ID NO: 51) | | |
| REP 2056 | TCTCTCTCTCTCTCTCTCTCTCTCTCTCTCTCTCTCTCTC | 40 | PS |
| | (SEQ ID NO: 52) | | |
| REP 2057 | AGAGAGAGAGAGAGAGAGAGAGAGAGAGAGAGAGAGAGAG | 40 | PS |
| | (SEQ ID NO: 53) | | |
| REP 2058 | TCTCCCAGCGTGCGCCAT | 18 | PS |
| | (SEQ ID NO: 54) | | |
| REP 2059 | NNNNNNNNNNNNNNNNNNNN | 20 | PS RNA |
| | (SEQ ID NO: 55) | | |
| REP 2060 | NNNNNNNNNNNNNNNNNNNNNNNNNNNNNN | 30 | PS RNA |
| | (SEQ ID NO: 56) | | |
| REP 2061 | ATATCCTTGTCGTATCCC | 18 | unmodified |
| | (SEQ ID NO: 57) | | |
| REP 2062 | ATATCCTTGTCGTATCCC | 18 | PS |
| | SEQ ID NO: 58 | | |
| REP 2063 | ATATCCTTGTCGTATCCC | 18 | PS, F-ANA |
| | (SEQ ID NO: 59) | | |
| REP 2064 | ATATCCTTGTCGTATCCC | 18 | PS, F-ANA |
| | (SEQ ID NO: 60) | | |
| REP 2065 | [AUAU]CCTTGTCGTA[UCCC] | 18 | PS, F-ANA, [2-Ome] |
| | SEQ ID NO: 61 | | |
| REP 2066 | [AUAU]CCTTGTCGTA[UCCC] | 18 | PS, F-ANA, [2-Ome] |
| | (SEQ ID NO: 62) | | |
| REP 2067 | [ATAT]CCTTGTCGTA[TCCC] | 18 | PS, F-ANA, [2-Ome] |
| | (SEQ ID NO: 63) | | |
| REP 2068 | ATATCCTTGTCGTATCCC | 18 | PS, F-ANA |
| | (SEQ ID NO: 64) | | |
| REP 2069 | NNNNNNNNNNNNNNNNNNNNNNNNNNNNNNNN | 32 | PS |
| | (SEQ ID NO: 65) | | |
| REP 2070 | NNNNNNNNNNNNNNNNNNNNNNNNNNNNNNNNNN | 34 | PS |
| | (SEQ ID NO: 66) | | |
| REP 2071 | NNNNNNNNNNNNNNNNNNNNNNNNNNNNNNNNNNNN | 36 | PS |
| | (SEQ ID NO: 67) | | |
| REP 2072 | NNNNNNNNNNNNNNNNNNNNNNNNNNNNNNNNNNNNNN | 38 | PS |
| | (SEQ ID NO: 68) | | |
| REP 2073 | GGAGGCTAGAAGGAGAGAGATGGGTGCGAGAGCGTCAGTA | 40 | PS |
| | (SEQ ID NO: 69) | | |
| REP 2074 | GTGGTGGGTGGGTGGGT | 17 | Unmodified/PS |
| | (SEQ ID NO: 70) | | |
| REP 2075 | GTGGTGGTGGTGTTGGTGGTGGTTTGGGGGGTGGGG | 36 | PS |
| | (SEQ ID NO: 71) | | |
| REP 2076 | GTGGTGGTGGTGTTGGTGGTGGTTTG | 26 | PS |
| | (SEQ ID NO: 72) | | |
| REP 2077 | GTGGTGGGTGGGTGGGTGGTGGGTGGTGGTTGTGGGTGGGTGG | 45 | PS |
| | TG | | |
| | (SEQ ID NO: 73) | | |
| REP 2078 | NNNNNNNNNNNNNNNNNN-C12-NNNNNNNNNNNNNNNNNNN | 37 (+1X3) | PS |
| | (SEQ ID NO: 12) (SEQ ID NO: 74) | | |
| REP 2079 | NNNNNNN-C12-NNNNNNNN-C12-NNNNNNNN-C12-NNNNNNNN | 31 (+3X3) | PS |
| | (SEQ ID NO: 75) (SEQ ID NO: 76) (SEQ ID NO: 76) (SEQ ID NO: 76) | | |
| REP 2080 | NNNNNNNNNN | 10 | PS/2-Ome gapmer |
| | (SEQ ID NO: 77) | | |
| REP 2081 | NNNNNNNNNNNNNNNNNNNN | 20 | PS/2-Ome gapmer |
| | (SEQ ID NO: 78) | | |
| REP 2082 | NNNNNNNNNN | 10 | PS/2-Ome full |
| | (SEQ ID NO: 79) | | |
| REP 2083 | NNNNNNNNNNNNNNNNNNNN | 20 | PS/2-Ome full |
| | (SEQ ID NO: 80) | | |
| REP 2084 | NNNNNNNNNN | 10 | 2-Ome full |
| | (SEQ ID NO: 81) | | |
| REP 2085 | NNNNNNNNNNNNNNNNNNNN | 20 | 2-Ome full |
| | (SEQ ID NO: 82) | | |
| REP 2086 | NNNNNNNNNNNNNNNNNNNNNNNNNNNNNNNNNNNNNN | 40 | 2-Ome full |
| | NN | | |
| | (SEQ ID NO: 83) | | |
| REP 2088 | NNNNNN | 6 | no modification |
| | (SEQ ID NO: 84) | | |
| REP 2089 | NNNNNN | 6 | 2-Ome full |
| | (SEQ ID NO: 85) | | |
| REP 2090 | NNNNNN | 6 | PS/2-Ome full |
| | (SEQ ID NO: 86) | | |
| REP 2091 | NNNNNNNNNNN | 11 | PS |
| | (SEQ ID NO: 87) | | |
| REP 2092 | NNNNNNNNNNNN | 12 | PS |
| | (SEQ ID NO: 88) | | |
| REP 2093 | NNNNNNNNNNNNN | 13 | PS |
| | (SEQ ID NO: 89) | | |
| REP 2094 | NNNNNNNNNNNNNN | 14 | PS |
| | (SEQ ID NO: 90) | | |
| REP 2095 | NNNNNNNNNNNNNNN | 15 | PS |
| | (SEQ ID NO: 91) | | |
| REP 2096 | NNNNNNNNNNNNNNNN | 16 | PS |
| | (SEQ ID NO: 92) | | |
| REP 2097 | NNNNNNNNNNNNNNNNN | 17 | Ps |
| | (SEQ ID NO: 93) | | |
| REP 2098 | NNNNNNNNNNNNNNNNNN | 18 | PS |
| | (SEQ ID NO: 94) | | |
| REP 2099 | NNNNNNNNNNNNNNNNNNN | 19 | PS |
| | (SEQ ID NO: 95) | | |
| REP 2100 | NNNNNNNNNNNNNNNNNNNNNNNNN | 25 | PS |
| | (SEQ ID NO: 96) | | |
| REP 2101 | NNNNNNNNNNNNNNNNNNNNNNNNNNNN | 28 | PS |
| | (SEQ ID NO: 97) | | |
| REP 2102 | NNNNNNNNNNNNNNNNNNNNNNNNNNNNNNNNNNNNNNNNNN | 42 | Ps |
| | (SEQ ID NO: 98) | | |
| REP 2103 | NNNNNNNNNNNNNNNNNNNNNNNNNNNNNNNNNNNNNNNNNNNN | 50 | PS |
| | NNNNNN | | |
| | (SEQ ID NO: 99) | | |
| REP 2104 | GTTCTCGCTGGTGAGTTTCA | 20 | PS |
| | (SEQ ID NO: 100) | | |
| REP 2105 | TCTCCCAGCGTGCGCCAT | 18 | PS |
| | (SEQ ID NO: 101) | | |
| Rep 2106 | NNNNNNNNNNNNNNNNNNNNNNNNNNNNNNNNNNNNNNNN | 40 | unmodified/PS |
| | (SEQ ID NO: 102) | | gapmer |
| Rep 2107 | NNNNNNNNNNNNNNNNNNNNNNNNNNNNNNNNNNNNNN | 40 | full 2-O-Me/full PS |
| | NN | | |
| | (SEQ ID NO: 103) | | |
| Rep 2108 | NNNNNNNNNNNNNNNNNNNNNNNNNNNNNN | 30 | full 2-O-Me/full PS |
| | (SEQ ID NO: 104) | | |
| Rep 2109 | NNNNNNNNNNNNNNNNNNNNNNNNNNNNNNNNNNNNNNNNNNNN | 50 | full 2-O-Me/full PS |
| | NNNNNN | | |
| | (SEQ ID NO: 105) | | |
| Rep 2110 | CCCCCCCCCCCCCCCCCCCCCCCCCCCCCCCCCCCCCCCC | 40 | unmodified |
| | (SEQ ID NO: 106) | | |
| Rep 2111 | TCGTCGTTTTGTCGTTTTGTCGTT | 24 | CPG 7909/PS |
| | (SEQ ID NO: 107) | | |
| Rep 2112 | CCCCCCCCCCCCCCCCCCCCCCCCCCCCCCCCCCCCCCCC | 40 | PS/each C = 5′- |
| | (SEQ ID NO: 108) | | methyl cytosine |
| Rep 2113 | NNNNNNNNNNNNNNNNNNNNNNNNNNNNNNNNNNNNNNNN | 40 | PS/except now N = |
| | (SEQ ID NO: 109) | | A, G, C or I (inosine) |
| Rep 2114 | IIIIIIIIIIIIIIIIIIIIIIIIIIIIIIIIIIIIIIII | 40 | PS |
| | (SEQ ID NO: 110) | | |
| Rep 2115 | UUUUUUUUUUUUUUUUUUUUUUUUUUUUUUUUUUUUUUUT | 40 | poly 4-thio dU/ |
| | (SEQ ID NO: 111) | | unmodified |
| Rep 2116 | UUUUUUUUUUUUUUUUUUUUUUUUUUUUUUUUUUUUUUUT | 40 | poly 4-thio dU/ |
| | (SEQ ID NO: 112) | | phosphorothiaote |
| Rep 2119 | TCGTCGTTTTCGGCGGCCGCCG | 22 | ODN 10101 |
| | (SEQ ID NO: 113) | | |
| Rep 2120 | NNNNNNNNNNNNNNNNNNNNNNNNNNNNNNNNNNNNNNNN | 40 | PS/each N = 5′- |
| | (SEQ ID NO: 114) | | methyl cytosine |
| Rep 2121 | NNNNNNNNNNNNNNNNNNNNNNNNNNNNNNNNNNNNNNN | 39 | PS |
| | (SEQ ID NO: 115) | | |
| Rep 2122 | NNNNNNNNNNNNNNNNNNNNNNNNNNNNNNNNNNNNN | 37 | PS |
| | (SEQ ID NO: 116) | | |
| Rep 2123 | NNNNNNNNNNNNNNNNNNNNNNNNNNNNNNNNNNN | 35 | PS |
| | (SEQ ID NO: 117) | | |
| Rep 2124 | NNNNNNNNNNNNNNNNNNNNNNNNNNNNNNNNN | 33 | PS |
| | (SEQ ID NO: 118) | | |
| Rep 2125 | NNNNNNNNNNNNNNNNNNNNNNNNNNNNNNN | 31 | PS |
| | (SEQ ID NO: 119) | | |
| Rep 2126 | CCCCCCCCCCCCCCCCCCCC | 20 | PS |
| | (SEQ ID NO: 120) | | |
| Rep 2127 | CCCCCCCCCCCCCCCCCCCCCCCCCCCCCC | 30 | PS |
| | (SEQ ID NO: 121) | | |
| Rep 2128 | CCCCCCCCCCCCCCCCCCCCCCCCCCCCCCCCCCCCCCCCCCCC | 50 | PS |
| | CCCCCC | | |
| | (SEQ ID NO: 122) | | |
| Rep 2129 | CCCCCCCCCCCCCCCCCCCCCCCCCCCCCCCCCCCCCCCCCCCC | 60 | PS |
| | CCCCCCCCCCCCCCCC | | |
| | (SEQ ID NO: 123) | | |

### EXAMPLE 1

### Clotting Factor/Randomer Interaction is Dependent on Randomer Length and Sulfur Modification

The effects of various lengths and chemical modifications of randomers on the strength of their interaction (k_{d}) with thrombin and fibrinogen were assessed using a gradient of protein concentrations (see Table 1). We observed that in general the binding to thrombin and fibronectin was more potent as the length of the randomer increased. We also noted that randomers which were not sulfur modified (phosphorothioated) but stabilized by the presence of a 2′-O-methyl group on each ribose (REP 2086, SEQ ID NO: 83) had a much weaker interactions with both thrombin and fibronectin, implying that sulfur modification (i.e. phosphorothioation) is essential for high affinity interaction with these clotting factors.

TABLE 1
Interaction of various randomers with thrombin and fibrinogen

| | | Kd for randomer - | Kd for randomer - |
|---|---|---|---|
| | | thrombin | fibrinogen |
| Randomer | SEQ ID NO | interaction (μM) | interaction (μM) |
| REP 2003 | SEQ ID NO: 3 | 0.14 | 0.081 |
| REP 2004 | SEQ ID NO: 4 | 0.049 | 0.018 |
| REP 2006 | SEQ ID NO: 6 | 0.022 | 0.0036 |
| REP 2007 | SEQ ID NO: 7 | 0.036 | 0.0056 |
| REP 2107 | SEQ ID NO: 103 | 0.012 | 0.019 |
| REP 2086 | SEQ ID NO: 83 | No interaction | No interaction |

Since it is demonstrated that sulfur modified randomers interact with at least two proteins involved in the clotting process, this length and sulfur requirement for high affinity binding suggests that any modification which increases the hydrophobicity of oligonucleotides will be beneficial for anti-clotting activity. Moreover, randomers of larger sizes (especially greater than 20 bases) may have a greater activity than smaller randomers.

### EXAMPLE 2

### Therapeutic Sulfur-Modified ONs with Increased pH Resistance, Lower Serum Protein Binding and Superior Nuclease Resistance

The effect of combining unmodified linkages, phosphorothiate linkages, 2′-O methyl modified riboses and unmodified ribonucleotides on the serum protein interaction and chemical stability of a 40 base randomer ON, were assessed in order to determine if they can be used as a therapeutic agent.

All randomers were prepared using standard solid phase, batch synthesis at the University of Calgary Core DNA Services lab on a 1 or 15 uM synthesis scale, deprotected and desalted on a 50 cm Sephadex® G-25 column.

To determine serum protein interaction, a phosphorothioate randomer labeled at the 3′ end with FITC (the bait) is diluted to 2 nM in assay buffer (10 mM Tris, pH7.2, 80 mM NaCl, 10 mM EDTA, 100 mM b-mercaptoethanol and 1% tween® 20). This oligonucleotide is then mixed with the appropriate amount of non heat-inactivated FBS. Following randomer-FBS interaction, the complexes are challenged with various unlabelled randomers to assess their ability to displace the bait from its complex. Displaced bait is measured by fluorescence polarization. The displacement curve was used to determine Kd.

pH resistance was determined by incubation of randomers in PBS adjusted to the appropriate pH with HCl. 24 hours after incubation, samples were neutralized with 1M TRIS, pH 7.4 and run on denaturing acryalmide gels and visualized following EtBr staining.

For these experiments, the behaviours of different modified ON randomers were compared: REP 2006 (SEQ ID NO: 6), REP 2024 (SEQ ID NO: 24), REP 2107 (SEQ ID NO: 103), REP 2086 (SEQ ID NO: 83) and REP 2060 (SEQ ID NO: 56).

The relative affinity of these ON randomers for serum proteins was determined as described above. The results of these experiments showed that REP 2107 (SEQ ID NO: 103) has a lower affinity to serum proteins than REP 2006 (SEQ ID NO: 6) or REP 2024 (SEQ ID NO: 24) (Table 2) and that there was no interaction detected between REP 2086 (SEQ ID NO: 83) and serum proteins. Moreover, at saturation of competition, REP 2107 (SEQ ID NO: 103) was less effective at displacing bound bait than REP 2006 (SEQ ID NO: 6) or REP 2024 (SEQ ID NO: 24) (Table 3 in this example).

TABLE 2
Serum protein affinity of various randomers

| Randomer | SEQ ID NO | Kd (μM) (FBS) |
|---|---|---|
| 2006 | SEQ ID NO: 6 | 0.013 |
| 2024 | SEQ ID NO: 24 | 0.013 |
| 2107 | SEQ ID NO: 103 | 0.027 |
| 2086 | SEQ ID NO: 83 | no binding |

TABLE 3
Displacement of bait randomer at saturation

| Randomer | SEQ ID NO | % displaced bait |
|---|---|---|
| 2006 | SEQ ID NO: 6 | 75 |
| 2024 | SEQ ID NO: 24 | 80 |
| 2107 | SEQ ID NO: 103 | 60 |
| 2086 | SEQ ID NO: 83 | no displacement |

The pH stability of these randomers in the range of pH 1 to pH 7 over 24 hours of incubation at 37° C. was tested. While REP 2006 (SEQ ID NO: 6) and REP 2024 (SEQ ID NO: 24) showed significant degradation at pH 3 and complete degradation at pH 2.5. REP 2107 (SEQ ID NO: 103), REP 2086 (SEQ ID NO: 83) and REP 2060 (SEQ ID NO: 56) were completely stable at pH 1 after 24 h of incubation.

It was demonstrated in the present invention that the incorporation of 2′-O methyl modifications in a sulfured-modified PS-ON randomers lowers serum protein binding and improve low pH resistance. The fully 2′-O-methylated, fully phosphorothioated randomer (REP 2107, SEQ ID NO: 103) has a weaker interaction with serum proteins and shows a significantly increased resistance to low pH induced hydrolysis.

40 mer randomers of various chemistries were assessed for their ability to resist degradation by various nucleases for 4 hours at 37° C. (Table 4 in this example). While most chemistries exhibited resistance to more than one nuclease, only REP 2107 (SEQ ID NO: 103) was resistant to all four nucleases tested. It is important to note that REP 2024 SEQ ID NO: 24; which has 2′-O methyl modifications at the 4 riboses at each end of the molecule) showed the same resistance profile as its parent molecule REP 2006 (SEQ ID NO: 6), being sensitive to S1 nuclease degradation while 2107 (SEQ ID NO: 103; fully 2′-O methyl modified) was resistant to this enzyme. These results suggest that fully 2′-O methyl modified and fully sulfured ON will be the most effective of the tested oligonucleotides in resisting degradation by nucleases.

TABLE 4
Resistance to various nucleases by different randomer chemistries

| | Sensitive (S) or Resistant (R) after 4 h incubation at 37° C. | | | |
|---|---|---|---|---|
| | Phosphodiesterase | S1 Nucleas | | Exonuclease I |
| | II | (Fermentas | Bal 31 | (NEB |
| Randomer | (Sigma P9041) | #EN0321) | (NEB M0213S) | M0293S) |
| REP 2015 | S | S | S | S |
| (SEQ ID NO: 15) | | | | |
| REP 2107 | R | R | R | R |
| (SEQ ID NO: 103) | | | | |
| REP 2006 | R | S | R | R |
| (SEQ ID NO: 6) | | | | |
| REP 2086 | R | R | S | R |
| (SEQ ID NO: 83) | | | | |
| REP 2024 | R | S | R | R |
| (SEQ ID NO: 24) | | | | |

### EXAMPLE 3

### Therapeutic Sulfur Modified Polypyrimidine ONs Exhibit Acid and Nuclease Resistance

To determine the extent of acid resistance of ONs that can be used as therapeutic agents, various 40 base ONs having different chemistries and/or sequences are incubated in PBS buffered to different pH values for 24 hours at 37° C. The degradation of these ONs was assessed by urea-polyacryamide gel electrophoresis (Table 5).

The results of these studies show that randomer ONs (containing both pyrimidine and purine nucleotides) are resistant to acidic pH only when they were fully 2′-O-methylated. Our data indicated that even partially 2′-O-methylated ONs (gapmers, REP 2024, SEQ ID NO: 24) do not display any significant increase in acid resistance compared to fully phosphorothioated ONs. Even fully phosphorothioated randomers show no increased pH resistance compared to unmodified ONs. In contrast, it was noted that the phosphorothioated 40mer ONs containing only the pyrimidine nucleotides cytosine (polyC, REP 2031, SEQ ID NO: 31) or thymidine (polyT, REP 2030, SEQ ID NO: 30) or the polyTC heteropolymer (REP 2056, SEQ ID NO: 52) had equivalent acid resistance compared to the fully 2′-O-methylated randomers whether phosphorothioated (REP 2107, SEQ ID NO: 103) or not (REP 2086, SEQ ID NO: 83). Contrary to the results for the polypyrimidine oligonucleotides, phosphorothioated oligonucleotides containing only the purine nucleotide adenosine (polyA, REP 2029, SEQ ID NO: 29) or any adenosine or guanosine nucleotides (REP 2033, SEQ ID NO: 33; REP 2055, SEQ ID NO: 51; REP 2057, SEQ ID NO: 53) showed no greater acid resistance compared to unmodified DNA.

These results are significant because the preferred way described in the prior art to achieve greater acid resistance compared to phosphorothioated ONs was to add 2′-O-methyl modifications (or other 2′-ribose modifications) or other modifications. The present data demonstrates that the 2′-O-methyl ribose modification or other 2′-ribose modifications are not required if the ON is a polypyrimidine (i.e. contains only pyrimidine nucleotides [e.g. homopolymers of cytosine or thymidine or a heteropolymer of cytosines and thymidines]) to achieve pH resistance. The presence of purines (A or G) even in the presence of pyrimidines, can render ONs acid labile.

TABLE 5
Acid stability of various 40 mer ONs

| | | | stability to various pHs after 24 h at 37° C. | | | | | | |
|---|---|---|---|---|---|---|---|---|---|
| ON name | sequence | modification | 1 | 2 | 2.5 | 3 | 4 | 5 | 7 |
| REP 2015 | randomer | none | − | − | −/+ | + | +++ | +++ | +++ |
| (SEQ ID NO: 15) | | | | | | | | | |
| REP 2006 | randomer | PS | − | − | −/+ | + | +++ | +++ | +++ |
| (SEQ ID NO: 6) | | | | | | | | | |
| REP 2086 | randomer | 2′OMe | +++ | +++ | +++ | +++ | +++ | +++ | +++ |
| (SEQ ID NO: 83) | | | | | | | | | |
| REP 2107 | randomer | PS, 2′OMe | +++ | +++ | +++ | +++ | +++ | +++ | +++ |
| (SEQ ID NO: 103) | | | | | | | | | |
| REP 2024 | randomer | PS, 2′OMe | − | − | −/+ | + | +++ | +++ | +++ |
| (SEQ ID NO: 24) | | gapmer | | | | | | | |
| REP 2031 | polyC | PS | +++ | +++ | +++ | +++ | +++ | +++ | +++ |
| (SEQ ID NO: 31) | | | | | | | | | |
| REP 2030 | polyT | PS | +++ | +++ | +++ | +++ | +++ | +++ | +++ |
| (SEQ ID NO: 30) | | | | | | | | | |
| REP 2029 | polyA | PS | − | − | − | − | ++ | +++ | +++ |
| (SEQ ID NO: 29) | | | | | | | | | |
| REP2033 | polyTG | PS | − | − | − | − | ++ | +++ | +++ |
| (SEQ ID NO: 33) | | | | | | | | | |
| REP 2055 | polyAC | PS | − | − | − | − | ++ | +++ | +++ |
| (SEQ ID NO: 51) | | | | | | | | | |
| REP 2056 | polyTC | PS | +++ | +++ | +++ | +++ | +++ | +++ | +++ |
| (SEQ ID NO: 52) | | | | | | | | | |
| REP 2057 | polyAG | PS | − | − | − | − | ++ | +++ | +++ |
| (SEQ ID NO: 53) | | | | | | | | | |

PII = phosphodiesterase II,
S1 = S1 nuclease,
Exo1 = Exonuclease 1,
PS = all linkages phosphorothioated,
2′OMe = all riboses are 2′O methylated.
+++ = no degradation,
++ = less than 5-% degradation,
−/+ = more than 90% degradation,
− = completely degraded.

To determine the extent of ON nucleotide composition and modifications on nuclease resistance, various 40 base ONs having different nucleotide compositions and modifications were incubated in the presence of various endo and exonucleases for 4 hours at 37 deg C. The degradation of these ONs was assessed by urea-polyacryamide gel electrophoresis.

The results of these studies showed that randomer ONs were resistant to all four enzymes tested (phosphodiesterase II, Sigma; S1 nuclease, Fermentas; Bal31, New England Biolabs; and exonuclease 1, New England Biolabs) only when they were fully phosphorothioated and fully 2′-O-methylated (Table 6). Omission of any of these modifications in randomers resulted in increased sensitivity to one or more of the nucleases tested. It was noted that the fully phosphorothioated, partially 2′-O-methylated randomer (REP 2024, SEQ ID NO: 24) was equivalent in nuclease resistance to REP 2006 (SEQ ID NO: 6), indicated that 2′-O-methylation may be required on each nucleotide of a phosphorothioated ON to achieve the optimal nuclease resistance. However, we noted that the phosphorothioated 40mer polypyrimidine poly cytosine (poly C, REP 2031, SEQ ID NO: 31) had equivalent nuclease resistance compared to the fully phosphorothioated, fully 2′O methylated randomer (REP 2107, SEQ ID NO: 103).

These results are significant because the prior art teaches that the preferred way to enhance nuclease resistance of phosphorothioated ONs is to add 2′-O-methyl modifications, other 2′-ribose modifications, or other modifications. This new data demonstrates that the 2′-O-methyl modification or other 2′-ribose modifications or any other modifications are not required to enhance nuclease resistance if the ON is fully phosphorothioated and consists of a homopolymer of cytosines.

TABLE 6
Nuclease resistance of various 40 mer ONs

| | | | Nuclease resistance after | | | |
|---|---|---|---|---|---|---|
| | | modi- | 4 h at 37° C. | | | |
| ON name | sequence | fication | PII | S1 | Bal 31 | Exo 1 |
| REP 2015 | randomer | none | − | − | − | − |
| (SEQ ID NO: 15) | | | | | | |
| REP 2006 | randomer | PS | +++ | − | ++++ | ++++ |
| (SEQ ID NO: 6) | | | | | | |
| REP 2086 | randomer | 2′OMe | ++++ | ++++ | − | ++++ |
| (SEQ ID NO: 83) | | | | | | |
| REP 2107 | randomer | PS, 2′OMe | ++++ | ++++ | ++++ | ++++ |
| (SEQ ID NO: | | | | | | |
| 103) | | | | | | |
| REP 2024 | randomer | PS, 2′OMe | ++++ | − | ++++ | ++++ |
| (SEQ ID NO: 24) | | gapmer | | | | |
| REP 2031 | polyC | PS | ++++ | ++++ | ++++ | ++++ |
| (SEQ ID NO: 31) | | | | | | |
| REP 2029 | Poly A | PS | − | − | ++++ | ++++ |
| (SEQ ID NO: 29) | | | | | | |
| REP 2030 | Poly T | PS | − | − | ++++ | ++++ |
| (SEQ ID NO: 30) | | | | | | |
| REP2033 | Poly TG | PS | + | − | ++++ | ++++ |
| (SEQ ID NO: 33) | | | | | | |
| REP 2055 | Poly AC | PS | + | − | ++++ | ++++ |
| (SEQ ID NO: 51) | | | | | | |
| REP 2056 | Poly TC | PS | + | − | ++++ | ++++ |
| (SEQ ID NO: 52) | | | | | | |
| REP 2057 | Poly AG | PS | ++ | − | ++++ | ++++ |
| (SEQ ID NO: 53) | | | | | | |

PII = phosphodiesterase II,
S1 = S1 nuclease,
Exo1 = Exonuclease 1,
PS = all linkages phosphorothioated,
2′OMe = all riboses are 2′O methylated.
− = complete degradation,
++++ = no degradation,
PS = phosphorothioate,
2′OMe = 2′-O-methyl modification of the ribose.

These results demonstrate that sulfur modified ONs containing only pyrimidine nucleotides, including cytosine and/or thymidine and/or other pyrimidines are resistant to low pH and phosphorothioated ONs containing only cytosine nucleotides exhibit superior nuclease resistance, two important characteristics for oral administration of an ON. Thus, high pyrimidine nucleotide content of an ON is advantageous to provide resistance to low pH resistance and high cytosine content is advantageous to provide improved nuclease resistance. For example, in certain embodiments, the pyrimidine content of such an oligonucleotide is more than 50%, more than 60%, or more than 70%, or more than 80%, or more than 90%, or 100%. Furthermore, these results show the potential of a method of treatment using oral administration of a therapeutically effective amount of at least one pharmacologically acceptable ON composed of pyrimidine nucleotides. These results also show the potential of ONs containing high levels of pyrimidine nucleotides as a component of an ON formulation.

### EXAMPLE 4

### ONs Increase Partial Thromboplastin Time in Blood

To test the effect of ONs as anticoagulant agents, various ONs were tested for their ability to inhibit coagulation activity in human blood. All blood samples were drawn from the same subject (who was not fasting) using standard procedure and collection tubes. One minute after collection, 0.4 ml of compound dissolved in normal saline was added to 3.6 ml of blood, followed by 10 inversions within one minute of compound addition. Partial thromboplastin time (PTT) was measured using the standard clinical procedure. Table 7 shows the effects of ONs on PTT time.

TABLE 7
Effects of various ONs on PTT

| | Final ON | | |
|---|---|---|---|
| | concentration | | |
| | (mM) | | |
| | 0.01 | 0.005 | 0.001 |
| ON | PTT (sec.) | | |
| None (Normal saline) | 26.8 | | |
| REP 2004 (SEQ ID NO: 4) | 38.0 | 33.5 | 28.5 |
| REP 2006 (SEQ ID NO: 6) | 51.0 | 36.9 | 28.7 |
| REP 2107 (SEQ ID NO: 103) | 45.9 | 33.9 | 27.9 |
| REP 2031 (SEQ ID NO: 31) | 51.8 | 36.6 | 29.3 |

These data show that ONs can increase the PTT of human blood and indicate that they have potential as anticoagulant or anti-thrombotic agents.

### EXAMPLE 5

### Sulfur Modified ONs Can Interact with Snake Venom

The interaction of different sizes of fully degenerate phosphorothioate oligonucleotides (Table 8) with a variety of snake venom proteins (dendrotoxin and Bungarotoxin from the Green Mamba) were assessed by a fluorescence polarization-based binding assay.

TABLE 8
Kd for interaction with PS-ONs with snake venom

Kd (μM)

| Compound | SEQ ID NO | dendrotoxin | bungarotoxin |
|---|---|---|---|
| REP 2003 | SEQ ID NO: 3 | 28.57 | 95 |
| REP 2006 | SEQ ID NO: 6 | 1.93 | 24.4 |
| REP 2107 | SEQ ID NO: 103 | 0.8 | 20.3 |
| REP 2031 | SEQ ID NO: 31 | 0.21 | 22.8 |

These data show the size dependent interaction of PS-ONs with snake venom, suggesting that longer PS-ONs will be more effective as anti-venom agents.

### EXAMPLE 6

### Apolipoprotein B/ON Interaction is Dependent on ON Length and Sulfur Modification

The effects of various lengths and chemical modifications of ONs on the strength of their interaction (K_{d}) with Apolipoprotein B (ApoB) were assessed using fluorescence polarization (Table 9). It was observed that in general the binding to ApoB was more potent as the length of the randomer increased. It was also noted that randomers which were not sulfur modified (phosphorothioated) but stabilized by the presence of a 2′-O-methyl group on each ribose (REP 2086, SEQ ID NO: 83) had no interaction with ApoB, implying that sulfur modification (i.e. phosphorothioation) is also essential for high affinity interaction with ApoB.

TABLE 9
Interaction of various randomers with ApoB

| | K_{d} for randomer - |
|---|---|
| Randomer | ApoB interaction (μM) |
| REP 2032 6mer PS-ON | No binding |
| randomer (SEQ ID NO: 32) | |
| REP 2003 (SEQ ID NO: 3) | Minimal binding |
| REP 2004 (SEQ ID NO: 4) | 0.00275 |
| REP 2006 (SEQ ID NO: 6) | 0.00135 |
| REP 2007 (SEQ ID NO: 7) | 0.00170 |
| REP 2086 (SEQ ID NO: 83) | No binding |
| REP 2107 (SEQ ID NO: 103) | 0.00123 |

This length and sulfur requirement for high affinity binding with ApoB suggests that these compounds may be useful agents for treating hypercholesterolemia. It also suggests that any other sulfur modifications may confer similar therapeutic activity.

### EXAMPLE 7

### Tests for Determining if an ON has Sequence-Independent Therapeutic Activity

The present invention discloses that sulfur modified ONs have potential anti-thrombotic, anti-cholesterol, anti-dyslipidemia, anti-osteoporosis and anti-venom activity. Moreover, it is shown that the protein binding activity of ONs to therapeutically relevant proteins in each case is sequence-independent, size dependent and dependent on sulfur modifications and is active in vivo. Of course any one skilled in the art could prepare sequence-specific ONs, for example an ON, longer than the established optimal length for antisense, which targets the mRNA of one of the proteins described herein. However such an ON would have benefited from the ON modifications and properties described herein and the fact that it was demonstrated herein that the activity of such a modified ON is sequence independent and size dependent. An ON shall be considered to have sequence-independent activity if it meets the criteria of any one of the 2 tests outlined below. An ON having a reasonable part of its function due to a sequence-independent activity shall be considered to benefit from the inventions described herein. Proteins used in these tests are involved in a disease described herein.

### TEST #1

### Effect of Partial Degeneracy of a Candidate ON on its Activity

This test serves to measure the protein interaction of a candidate ON sequence when part of its sequence is made degenerate. If the degenerate version of the candidate ON having the same chemistry retains its activity as described below, it is deemed to have sequence-independent activity. Candidate ONs will be made degenerate according to the following rule:

L=the number of bases in the candidate ON;
X=the number of bases on each end of the oligo to be made degenerate (but having the same chemistry as the candidate ON);
If L is even, then X=integer (L/4);
If L is odd, then X=integer ((L+1)/4);
X must be equal to or greater than 4.

The protein binding activity of the candidate and partially degenerate ON shall be determined using fluorescence polarization (FP) using 3′ FITC-labelled oligonucleotides (the candidate and its partially degenerate version) with a rigid 6-carbon spacer (and any of the purified proteins described herein. The binding (Kd) shall be generated using a minimum of seven concentrations of purified protein using a fixed (2-5 nM) concentration of FITC-labelled ON and a concentration range for each protein as described above to demonstrate interaction with REP 2006 (SEQ ID NO: 6), with three or more points in the linear range of the dose response curve. The Kd shall represent the quantity of protein which results in a 50% binding to the protein in question (or 50% of increase in FP compared to the increase in FP seen under saturated protein-binding conditions). Using these tests, the Kd of the candidate ON with any of these proteins shall be compared to its degenerate counterpart for the same protein. If the Kd of the partially degenerate ON for a particular protein is less than 5-fold greater than the original candidate ON for the same protein (based on minimum triplicate measurements, standard deviation not to exceed 15% of mean) then the candidate ON shall be deemed to have sequence independent activity.

### TEST #2

### Broad Spectrum Activity of a Candidate ON

Since it was demonstrated that the general, sequence independent properties of ONs required for interaction with all the different proteins described herein are highly similar (e.g. size dependent, sequence-independent and dependent on sulfur modifications, then any candidate ON designed to be therapeutically specific, either as an antisense or as a sequence-specific aptamer should only interact with its intended target (e.g. only one of the proteins described herein). This test serves to compare the protein binding activity of the candidate ON with all the proteins identified herein. The binding (Kd) of the 3′FITC-labeled candidate ON with each protein shall be performed as described above in test #1. The candidate ON shall be considered to interact with any specific protein if there is a greater than 100% increase in the FP relative to the candidate ON in the absence of protein. Thus, if the candidate ON interacts with more than one of the proteins described above, then the candidate ON shall be deemed to have sequence-independent activity benefiting from the art taught by this patent.

### Thresholds Used in These Tests

The purpose of these tests are to determine by a reasonable analysis, if ONs benefit from or utilize the sequence-independent protein binding properties of ONs which we have described herein and is acting with sequence-independent activity. Of course anyone skilled in the art will realize that, given the inherent variability of all testing methodologies, a determination of differences in protein binding activity between two compounds may not be reliably concluded if the threshold is set at a 2 or 3 fold differences between the activities of said compounds. This is due to the fact that variations from experiment to experiment with the same compound in these assays can yield Kds which vary in this range. Thus, to be reasonably certain that real differences between the binding activities of two compounds (e.g. two ONs) exist, we have set a threshold of at least a 5-fold difference between the Kds s of said compounds. This threshold ensures the reliability of the assessment of the above mentioned tests.

The threshold described in test 1 is the default threshold. If specifically indicated, other thresholds can be used in test. Thus for example, if specifically indicated, the threshold for determining whether an ON is acting with sequence-independent activity can be any of 10-fold, 8-fold, 6-fold, 5-fold, 4-fold, 3-fold, 2-fold, 1.5-fold, or equal.

Similarly, though the default is that satisfying any one of the above 2 tests is sufficient, if specifically indicated, the ON can be required to satisfy both at a default threshold, or if specifically indicated, at another threshold(s) as indicated above.

### EXAMPLE 8

### ONs Interact with Proteins Involved in Metabolic Disorders

Certain proteins, including resistin and are known to be involved in metabolic disorders including without restriction the metabolic syndrome and obesity. Other proteins such as apolipoprotein E (Apo E) or low density lipoprotein complexes (LDL) are know to be involved in metabolic disorders including dyslipidemia such as hypercholesterolemia. To examine the interaction of ONs with these proteins, a completely degenerate (randomer) 40 mer phosphorothioated ON (REP 2006, SEQ ID NO: 6) or its polycytidylic analog (REP 2031, SEQ ID NO: 31) were fluorescently labelled at the 3′ end. The interaction of labelled REP 2006 (SEQ ID NO: 6) or REP 2031 (SEQ ID NO: 31) with these various proteins in solution was measured by fluorescence polarization (Table 10). Larger increases in the ΔmP (the increase in mP from the baseline value seen in the absence of protein) indicates stronger interactions and a ΔmP of zero indicate no interaction.

TABLE 10
REP 2006 (SEQ ID NO: 6) and REP 2031 (SEQ ID NO: 31) interact
with proteins involved in metabolic disorders

mP

| | REP 2006 | REP 2031 |
|---|---|---|
| Protein (complex) | (SEQ ID NO: 6) | (SEQ ID NO: 31) |
| None (baseline) | 70 | 50 |
| LDL | 207 | 115 |
| Apo E | 413 | 358 |
| resistin | 225 | Not tested |

These data demonstrate that ONs can interact with protein targets involved in metabolic disorders and may be effective therapeutic agents to treat these disorders.

### EXAMPLE 9

### ON Interaction with VLDL is Dependent on ON Length and Sulfur Modification

The effects of various lengths and chemical modifications of ONs on the strength of their interaction (K_{D}) with VLDL were assessed using fluorescence polarization (FP) using a gradient of target concentrations to determine the binding strength of each target to ON (Table 11). It was observed that in general, the binding of VLDL to ONs was more potent as the length of the ON increased. It was also noted that ONs which were not sulfur modified (phosphorothioated) but stabilized by the presence of a 2′-O-methyl group on each ribose (REP 2086, SEQ ID NO: 83) had a much weaker interaction with VLDL, implying that sulfur modification (i.e. phosphorothioation) and ON lengths 40 bases in length or greater is essential for high affinity interaction with VLDL.

TABLE 11
Interaction of various randomers with VLDL

| | | K_{D} VLDL - ON |
|---|---|---|
| ON | SEQ ID NO | interaction (ng/ml) |
| REP 2032 | SEQ ID NO: 32 | No binding |
| REP 2003 | SEQ ID NO: 3 | Minimal binding |
| REP 2004 | SEQ ID NO: 4 | 4.77 |
| REP 2006 | SEQ ID NO: 6 | 2.29 |
| REP 2007 | SEQ ID NO: 7 | 4.73 |
| REP 2086 | SEQ ID NO: 83 | No binding |
| REP 2031 | SEQ ID NO: 31 | 4.95 |
| REP 2107 | SEQ ID NO: 103 | 3.36 |

These data shows that ONs interact with VLDL in a size and sulfur dependent manner and may be used as therapeutic agents to treat dyslipidemia such as hypercholesterolemia and high level of VLDL.

### EXAMPLE 10

### ONs Interact with Cytokines Involved in Metabolic Disorders

Certain cytokines, including TNF-α, IL-1 and IL-6 are known to be involved in metabolic disorders including without restriction the metabolic syndrome and obesity. To examine the interaction of ONs with various cytokines, a completely degenerate (randomer) 40 mer phosphorothioated ON (REP 2006, SEQ ID NO: 6) was fluorescently labelled at the 3′ end (REP 2006-FL). The interaction of REP 2006-FL with various recombinant or immunopurified human cytokines in solution (at 0.1 μg/μl) was measured by fluorescence polarization (Table 12). Larger increases in the mP (a dimensionless unit describing the relative extent of fluorescence polarization) value above the baseline value seen with REP 2006-FL in the absence of cytokines indicates stronger interactions. No increase from the baseline indicates no interaction.

TABLE 12
REP 2006(SEQ ID NO: 12) interacts with cytokines involved in
metabolic disorders

| Cytokine | mP |
|---|---|
| None (baseline) | 72 |
| TNF-α | 226 |
| IL-1β | 256 |
| IL-6 | 425 |

These data demonstrate that ONs can interact with cytokines involved in metabolic disorders such as the metabolic syndrome and obesity and may be an effective therapeutic agent to treat these disorders.

### EXAMPLE 11

### ONs can Prevent Hypercholesterolemia and Obesity in vivo

To assess if ONs could prevent the onset of dyslipidenia, hypercholesterolemia, body fat accumulation, hyper triglyceridemia and obesity in hamsters fed a high fructose (HF) diet, REP 2031 (SEQ ID NO: 31), a 40mer fully phosphorothioated oligodeoxycytidylic acid was administered to animals on a HF diet by intraperitoneal injection 3 times a week for 4 weeks. Several parameters relating to hypercholesterolemia and obesity were monitored (Table 13).

TABLE 13
Effects of REP 2031 in HF fed hamsters

| | Normal | | |
|---|---|---|---|
| Parameter | chow diet | High Fructose Diet | |
| measured | Normal saline | Normal saline | REP 2031 10 mg/kg |
| initial | 93.5 ± 2.11 | 92.7 ± 2.36 | 93.47 ± 1.49 |
| weight (g) | | | |
| final | 116.5 ± 2.97 | 121.74 ± 3.33 | 116.02 ± 2.10 |
| weight (g) | | | |
| weight | 23.46 ± 3.02 | 28.2 ± 3.38 | 22.75 ± 2.13 |
| gain (g) | | | |
| eWAT fat | 0.5057 ± 0.030 | 0.5826 ± 0.0337 | 0.5261 ± 0.021 |
| pad weight | | | |
| (g) | | | |
| Cholesterol | 3.54 ± 0.304 | 4.432 ± 0.341 | 3.82 ± 0.215 |
| (mM) | | | |
| Triglycerides | 2.295 ± 0.045 | 2.379 ± 0.050 | 2.286 ± 0.032 |
| (mEq/l) | | | |

eWAT = epydidymal white adipose tissue

These results show that ON administration resulted in inhibition of weight gain, of body fat accumulation (eWAT), of increases in triglycerides and of hypercholesterolemia associated with a HF diet. Thus ONs can be used as therapeutic agent for dyslipidemia such as, but without restriction, hypercholesterolemia, hypertriglyceridemia and body fat accumulation; for obesity and for the metabolic syndrome.

### EXAMPLE 12

### ONs Interact with Different Apo E Isoforms

To see if oligonucleotides could interact with different Apo E isoforms, the interaction of FITC-labelled REP 2006 (SEQ ID NO: 6) and REP 2031 (SEQ ID NO: 31) with Apo E2, E3 and E4 was determined by fluorescence polarization. Dissociation constants (K_{D}) were determined from binding isotherms generated form several different concentrations of Apolipoprotein E isoforms.

TABLE 14
Interaction of REP 2006 and REP 2031 with Apo E isoforms

K_{D} (ng/ml)

| | REP 2006 | REP 2031 |
|---|---|---|
| Apo E isoform | (SEQ ID NO: 6) | (SEQ ID NO: 31) |
| E2 | 0.269 | 0.549 |
| E3 | 0.251 | 0.489 |
| E4 | 0.288 | 0.676 |

These data show that ONs interact with all Apo E isoforms equivalently and can be useful in the treatment of genetically related cholesterol related disorders.

1-125. (canceled)

126. A method for the prophylaxis or treatment in a subject of a disease or a condition selected from the group consisting of cholesterol related condition, dyslipidemia, obesity and the metabolic syndrome; comprising administering to a subject in need of such treatment a therapeutically effective amount of at least one pharmacologically acceptable oligonucleotide, wherein said oligonucleotide comprises at least one phosphorothioate linkage and at least 25 nucleotides and wherein the activity of said oligonucleotide occurs principally by a sequence independent mode of action.

127. The method of claim 126, wherein said oligonucleotide is at least 30 nucleotides in length.

128. The method of claim 126, wherein said oligonucleotide is at least 35 nucleotides in length.

129. The method of claim 126, wherein said oligonucleotide is at least 40 nucleotides in length.

130. The method of claim 126, wherein said oligonucleotide comprises a homopolymer sequence of at least 10 contiguous nucleotides selected from the group consisting of A, C, G, T, and any other synthetic or naturally-occurring base.

131. The method of claim 126, wherein said oligonucleotide is a homopolymer comprising nucleotides selected from the group consisting of A, C, T, and any other synthetic or naturally-occurring base.

132. The method of claim 126, wherein said oligonucleotide comprises a sequence of at least 10 contiguous nucleotides wherein said sequence is a repetitive sequence that alternates between two different nucleotides selected from the group consisting of A, T, G, C, and any other synthetic or naturally-occurring base.

133. The method of claim 126, wherein the entire sequence of said oligonucleotide is a repetitive sequence that alternates between two different nucleotides selected from the group consisting of A, T, G, C, and any other synthetic or naturally-occurring base

134. The method of claim 126, wherein said oligonucleotide is SEQ ID NO: 31

135. The method of claim 126, wherein said oligonucleotide is SEQ ID NO: 51

136. The method of claim 126, wherein said oligonucleotide comprises at least one additional modification other than a phosphorothioation to its chemical structure.

137. The method of claim 126, wherein said oligonucleotide comprises at least one 5-methylcytosine

138. The method of claim 126, wherein said oligonucleotide comprises at least one modification selected from the group consisting of 2′-O-methyl, 2′-methoxyethyl and 2′-fluoro.

139. The method of claim 126, wherein the sequence said oligonucleotide is not complementary to any equal length portion of a human genome sequence over the entire length of said equal length portion.

140. A therapeutic oligonucleotide formulation targeting a disease or a condition selected from the group consisting of cholesterol related condition, dyslipidemia, obesity and the metabolic syndrome comprising at least one oligonucleotide; wherein said oligonucleotide comprises at least one phosphorothioate linkage and at least 25 nucleotides in length; wherein the activity of said oligonucleotide occurs principally by a sequence independent mode of action.

141. The therapeutic oligonucleotide formulation of claim 140 wherein said oligonucleotide is a homopolymer comprising nucleotides selected from the group consisting of A, C, T, and any other synthetic or naturally-occurring base.

142. The therapeutic oligonucleotide formulation of claim 140 wherein the entire sequence of said oligonucleotide is a repetitive sequence that alternates between two different nucleotides selected from the group consisting of A, T, G, C, and any other synthetic or naturally-occurring base.

143. The therapeutic oligonucleotide formulation of claim 140 wherein the sequence said oligonucleotide is not complementary to any equal length portion of a human genome sequence over the entire length of said equal length portion.

144. A pharmaceutical composition comprising a therapeutically effective amount of at least one pharmacologically acceptable oligonucleotide formulation according to claim 140 and a pharmaceutically acceptable carrier.

145. A pharmaceutical composition comprising a therapeutically effective amount of at least one pharmacologically acceptable oligonucleotide formulation according to claim 141 and a pharmaceutically acceptable carrier.

146. A pharmaceutical composition comprising a therapeutically effective amount of at least one pharmacologically acceptable oligonucleotide formulation according to claim 142 and a pharmaceutically acceptable carrier.

147. A pharmaceutical composition comprising a therapeutically effective amount of at least one pharmacologically acceptable oligonucleotide formulation according to claim 143 and a pharmaceutically acceptable carrier.

148. A kit for the prophylaxis or treatment in a subject of a disease or a condition selected from the group consisting of cholesterol related condition, dyslipidemia, obesity and the metabolic syndrome, comprising a pharmaceutical composition as defined in claim 144 and instructions for use.


SEQUENCE LISTING (123 sequences)

SEQ ID NO: 1 - DNA, 22 bp, Artificial Sequence
  oligomer
gaagcgttcgcacttcgtccca

SEQ ID NO: 2 - DNA, 5 bp, Artificial Sequence
  oligomer
nnnnn

SEQ ID NO: 3 - DNA, 10 bp, Artificial Sequence
  oligomer
nnnnnnnnnn

SEQ ID NO: 4 - DNA, 20 bp, Artificial Sequence
  oligomer
nnnnnnnnnnnnnnnnnnnn

SEQ ID NO: 5 - DNA, 30 bp, Artificial Sequence
  oligomer
nnnnnnnnnnnnnnnnnnnnnnnnnnnnnn

SEQ ID NO: 6 - DNA, 40 bp, Artificial Sequence
  oligomer
nnnnnnnnnnnnnnnnnnnnnnnnnnnnnnnnnnnnnnnn

SEQ ID NO: 7 - DNA, 80 bp, Artificial Sequence
  oligomer
nnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 8 - DNA, 120 bp, Artificial Sequence
  oligomer
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 9 - DNA, 12 bp, Artificial Sequence
  oligomer
nnnnnnnnnnnn

SEQ ID NO: 10 - DNA, 14 bp, Artificial Sequence
  oligomer
nnnnnnnnnnnnnn

SEQ ID NO: 11 - DNA, 16 bp, Artificial Sequence
  oligomer
nnnnnnnnnnnnnnnn

SEQ ID NO: 12 - DNA, 18 bp, Artificial Sequence
  oligomer
nnnnnnnnnnnnnnnnnn

SEQ ID NO: 13 - DNA, 10 bp, Artificial Sequence
  oligomer
nnnnnnnnnn

SEQ ID NO: 14 - DNA, 20 bp, Artificial Sequence
  oligomer
nnnnnnnnnnnnnnnnnnnn

SEQ ID NO: 15 - DNA, 40 bp, Artificial Sequence; identical to SEQ ID NO: 6
  oligomer

SEQ ID NO: 16 - DNA, 10 bp, Artificial Sequence
  oligomer
tccgaagacg

SEQ ID NO: 17 - DNA, 20 bp, Artificial Sequence
  oligomer
acacctccgaagacgataac

SEQ ID NO: 18 - DNA, 40 bp, Artificial Sequence
  oligomer
ctacagacatacacctccgaagacgataacactagacata

SEQ ID NO: 19 - DNA, 10 bp, Artificial Sequence
  oligomer
cccccatgga

SEQ ID NO: 20 - DNA, 20 bp, Artificial Sequence
  oligomer
tacgacccccatggagcccc

SEQ ID NO: 21 - DNA, 40 bp, Artificial Sequence
  oligomer
tccagccgcatacgacccccatggagccccgccccggagc

SEQ ID NO: 22 - DNA, 40 bp, Artificial Sequence; identical to SEQ ID NO: 6
  oligomer

SEQ ID NO: 23 - DNA, 40 bp, Artificial Sequence; identical to SEQ ID NO: 6
  oligomer

SEQ ID NO: 24 - DNA, 40 bp, Artificial Sequence; identical to SEQ ID NO: 6
  oligomer

SEQ ID NO: 25 - DNA, 44 bp, Artificial Sequence
  oligomer
agagnnnnnnnnnnnnnnnnnnnnnnnnnnnnnnnnnnnnagag

SEQ ID NO: 26 - DNA, 40 bp, Artificial Sequence; identical to SEQ ID NO: 6
  oligomer

SEQ ID NO: 27 - DNA, 40 bp, Artificial Sequence; identical to SEQ ID NO: 6
  oligomer

SEQ ID NO: 28 - DNA, 40 bp, Artificial Sequence
  oligomer
gggggggggggggggggggggggggggggggggggggggg

SEQ ID NO: 29 - DNA, 40 bp, Artificial Sequence
  oligomer
aaaaaaaaaaaaaaaaaaaaaaaaaaaaaaaaaaaaaaaa

SEQ ID NO: 30 - DNA, 40 bp, Artificial Sequence
  oligomer
tttttttttttttttttttttttttttttttttttttttt

SEQ ID NO: 31 - DNA, 40 bp, Artificial Sequence
  oligomer
cccccccccccccccccccccccccccccccccccccccc

SEQ ID NO: 32 - DNA, 6 bp, Artificial Sequence
  oligomer
nnnnnn

SEQ ID NO: 33 - DNA, 40 bp, Artificial Sequence
  oligomer
tgtgtgtgtgtgtgtgtgtgtgtgtgtgtgtgtgtgtgtg

SEQ ID NO: 34 - DNA, 20 bp, Artificial Sequence
  oligomer
tgtgtgtgtgtgtgtgtgtg

SEQ ID NO: 35 - DNA, 10 bp, Artificial Sequence
  oligomer
tgtgtgtgtg

SEQ ID NO: 36 - DNA, 21 bp, Artificial Sequence
  oligomer
gcgtttgctcttcttcttgcg

SEQ ID NO: 37 - DNA, 25 bp, Artificial Sequence
  oligomer
ctctcgcacccatctctctccttct

SEQ ID NO: 38 - DNA, 21 bp, Artificial Sequence
  oligomer
ucgcacccatctctctccuuc

SEQ ID NO: 39 - DNA, 48 bp, Artificial Sequence
  oligomer
nnnnnnnnnnnnnnnnnnnnnnnnnnnnnnnnnnnnnnnnnnnnnnnn

SEQ ID NO: 40 - DNA, 68 bp, Artificial Sequence
  oligomer
nnnnnnnnnnnnnnnnnnnnnnnnnnnnnnnnnnnnnnnnnnnnnnnnnnnnnnnnnnnnnnnnnnnn

SEQ ID NO: 41 - DNA, 60 bp, Artificial Sequence
  oligomer
nnnnnnnnnnnnnnnnnnnnnnnnnnnnnnnnnnnnnnnnnnnnnnnnnnnnnnnnnnnn

SEQ ID NO: 42 - DNA, 30 bp, Artificial Sequence
  oligomer
cccccggggggtgtgtttcggggggggccc

SEQ ID NO: 43 - DNA, 40 bp, Artificial Sequence
  oligomer
ccgcgcgcgacccccggggggtgtgtttcggggggggccc

SEQ ID NO: 44 - DNA, 40 bp, Artificial Sequence
  oligomer
cgactggtgagtacgccaaaaattttgactagcggaggct

SEQ ID NO: 45 - RNA, 16 nt, Artificial Sequence
  oligomer
aguagaaacaagggug

SEQ ID NO: 46 - DNA, 15 bp, Artificial Sequence
  oligomer
cacccugcuuuugcu

SEQ ID NO: 47 - DNA, 16 bp, Artificial Sequence
  oligomer
agtagaaacaagggtg

SEQ ID NO: 48 - DNA, 15 bp, Artificial Sequence
  oligomer
caccctgcttttgct

SEQ ID NO: 49 - RNA, 71 nt, Artificial Sequence
  oligomer
aguagaaacaagggugnnnnnnnnnnnnnnnnnnnnnnnnnnnnnnnnnnnnnnnncacccugcuuuugcu

SEQ ID NO: 50 - DNA, 71 bp, Artificial Sequence
  oligomer
agtagaaacaagggtcnnnnnnnnnnnnnnnnnnnnnnnnnnnnnnnnnnnnnnnncaccctgcttttgct

SEQ ID NO: 51 - DNA, 40 bp, Artificial Sequence
  oligomer
acacacacacacacacacacacacacacacacacacacac

SEQ ID NO: 52 - DNA, 40 bp, Artificial Sequence
  oligomer
tctctctctctctctctctctctctctctctctctctctc

SEQ ID NO: 53 - DNA, 40 bp, Artificial Sequence
  oligomer
agagagagagagagagagagagagagagagagagagagag

SEQ ID NO: 54 - DNA, 18 bp, Artificial Sequence
  oligomer
tctcccagcgtgcgccat

SEQ ID NO: 55 - RNA, 20 nt, Artificial Sequence
  oligomer
nnnnnnnnnnnnnnnnnnnn

SEQ ID NO: 56 - RNA, 30 nt, Artificial Sequence
  oligomer
nnnnnnnnnnnnnnnnnnnnnnnnnnnnnn

SEQ ID NO: 57 - DNA, 18 bp, Artificial Sequence
  oligomer
atatccttgtcgtatccc

SEQ ID NO: 58 - DNA, 18 bp, Artificial Sequence
  oligomer
atatccttgtcgtatccc

SEQ ID NO: 59 - DNA, 18 bp, Artificial Sequence
  oligomer
atatccttgtcgtatccc

SEQ ID NO: 60 - DNA, 18 bp, Artificial Sequence
  oligomer
atatccttgtcgtatccc

SEQ ID NO: 61 - DNA, 18 bp, Artificial Sequence
  oligomer
auauccttgtcgtauccc

SEQ ID NO: 62 - DNA, 18 bp, Artificial Sequence
  oligomer
auauccttgtcgtauccc

SEQ ID NO: 63 - DNA, 18 bp, Artificial Sequence
  oligomer
atatccttgtcgtatccc

SEQ ID NO: 64 - DNA, 18 bp, Artificial Sequence
  oligomer
atatccttgtcgtatccc

SEQ ID NO: 65 - DNA, 32 bp, Artificial Sequence
  oligomer
nnnnnnnnnnnnnnnnnnnnnnnnnnnnnnnn

SEQ ID NO: 66 - DNA, 34 bp, Artificial Sequence
  oligomer
nnnnnnnnnnnnnnnnnnnnnnnnnnnnnnnnnn

SEQ ID NO: 67 - DNA, 36 bp, Artificial Sequence
  oligomer
nnnnnnnnnnnnnnnnnnnnnnnnnnnnnnnnnnnn

SEQ ID NO: 68 - DNA, 38 bp, Artificial Sequence
  oligomer
nnnnnnnnnnnnnnnnnnnnnnnnnnnnnnnnnnnnnn

SEQ ID NO: 69 - DNA, 40 bp, Artificial Sequence
  oligomer
ggaggctagaaggagagagatgggtgcgagagcgtcagta

SEQ ID NO: 70 - DNA, 17 bp, Artificial Sequence
  oligomer
gtggtgggtgggtgggt

SEQ ID NO: 71 - DNA, 36 bp, Artificial Sequence
  oligomer
gtggtggtggtgttggtggtggtttggggggtgggg

SEQ ID NO: 72 - DNA, 26 bp, Artificial Sequence
  oligomer
gtggtggtggtgttggtggtggtttg

SEQ ID NO: 73 - DNA, 45 bp, Artificial Sequence
  oligomer
gtggtgggtgggtgggtggtgggtggtggttgtgggtgggtggtg

SEQ ID NO: 74 - DNA, 19 bp, Artificial Sequence
  oligomer
nnnnnnnnnnnnnnnnnnn

SEQ ID NO: 75 - DNA, 7 bp, Artificial Sequence
  oligomer
nnnnnnn

SEQ ID NO: 76 - DNA, 8 bp, Artificial Sequence
  oligomer
nnnnnnnn

SEQ ID NO: 77 - DNA, 10 bp, Artificial Sequence
  oligomer
nnnnnnnnnn

SEQ ID NO: 78 - DNA, 20 bp, Artificial Sequence
  oligomer
nnnnnnnnnnnnnnnnnnnn

SEQ ID NO: 79 - DNA, 10 bp, Artificial Sequence
  oligomer
nnnnnnnnnn

SEQ ID NO: 80 - DNA, 20 bp, Artificial Sequence
  oligomer
nnnnnnnnnnnnnnnnnnnn

SEQ ID NO: 81 - DNA, 10 bp, Artificial Sequence
  oligomer
nnnnnnnnnn

SEQ ID NO: 82 - DNA, 20 bp, Artificial Sequence
  oligomer
nnnnnnnnnnnnnnnnnnnn

SEQ ID NO: 83 - DNA, 40 bp, Artificial Sequence; identical to SEQ ID NO: 6
  oligomer

SEQ ID NO: 84 - DNA, 6 bp, Artificial Sequence
  oligomer
nnnnnn

SEQ ID NO: 85 - DNA, 6 bp, Artificial Sequence
  oligomer
nnnnnn

SEQ ID NO: 86 - DNA, 6 bp, Artificial Sequence
  oligomer
nnnnnn

SEQ ID NO: 87 - DNA, 11 bp, Artificial Sequence
  oligomer
nnnnnnnnnnn

SEQ ID NO: 88 - DNA, 12 bp, Artificial Sequence
  oligomer
nnnnnnnnnnnn

SEQ ID NO: 89 - DNA, 13 bp, Artificial Sequence
  oligomer
nnnnnnnnnnnnn

SEQ ID NO: 90 - DNA, 14 bp, Artificial Sequence
  oligomer
nnnnnnnnnnnnnn

SEQ ID NO: 91 - DNA, 15 bp, Artificial Sequence
  oligomer
nnnnnnnnnnnnnnn

SEQ ID NO: 92 - DNA, 16 bp, Artificial Sequence
  oligomer
nnnnnnnnnnnnnnnn

SEQ ID NO: 93 - DNA, 17 bp, Artificial Sequence
  oligomer
nnnnnnnnnnnnnnnnn

SEQ ID NO: 94 - DNA, 18 bp, Artificial Sequence
  oligomer
nnnnnnnnnnnnnnnnnn

SEQ ID NO: 95 - DNA, 19 bp, Artificial Sequence
  oligomer
nnnnnnnnnnnnnnnnnnn

SEQ ID NO: 96 - DNA, 25 bp, Artificial Sequence
  oligomer
nnnnnnnnnnnnnnnnnnnnnnnnn

SEQ ID NO: 97 - DNA, 28 bp, Artificial Sequence
  oligomer
nnnnnnnnnnnnnnnnnnnnnnnnnnnn

SEQ ID NO: 98 - DNA, 42 bp, Artificial Sequence
  oligomer
nnnnnnnnnnnnnnnnnnnnnnnnnnnnnnnnnnnnnnnnnn

SEQ ID NO: 99 - DNA, 50 bp, Artificial Sequence
  oligomer
nnnnnnnnnnnnnnnnnnnnnnnnnnnnnnnnnnnnnnnnnnnnnnnnnn

SEQ ID NO: 100 - DNA, 20 bp, Artificial Sequence
  oligomer
gttctcgctggtgagtttca

SEQ ID NO: 101 - DNA, 18 bp, Artificial Sequence
  oligomer
tctcccagcgtgcgccat

SEQ ID NO: 102 - DNA, 40 bp, Artificial Sequence; identical to SEQ ID NO: 6
  oligomer

SEQ ID NO: 103 - DNA, 40 bp, Artificial Sequence; identical to SEQ ID NO: 6
  oligomer

SEQ ID NO: 104 - DNA, 30 bp, Artificial Sequence; identical to SEQ ID NO: 5
  oligomer

SEQ ID NO: 105 - DNA, 50 bp, Artificial Sequence; identical to SEQ ID NO: 99
  oligomer

SEQ ID NO: 106 - DNA, 40 bp, Artificial Sequence; identical to SEQ ID NO: 31
  oligomer

SEQ ID NO: 107 - DNA, 24 bp, Artificial Sequence
  oligomer
tcgtcgttttgtcgttttgtcgtt

SEQ ID NO: 108 - DNA, 40 bp, Artificial Sequence; identical to SEQ ID NO: 31
  oligomer

SEQ ID NO: 109 - DNA, 40 bp, Artificial Sequence; identical to SEQ ID NO: 6
  oligomer

SEQ ID NO: 110 - DNA, 40 bp, Artificial Sequence; identical to SEQ ID NO: 6
  oligomer

SEQ ID NO: 111 - DNA, 40 bp, Artificial Sequence
  oligomer
uuuuuuuuuuuuuuuuuuuuuuuuuuuuuuuuuuuuuuut

SEQ ID NO: 112 - DNA, 40 bp, Artificial Sequence; identical to SEQ ID NO: 111
  oligomer

SEQ ID NO: 113 - DNA, 22 bp, Artificial Sequence
  oligomer
tcgtcgttttcggcggccgccg

SEQ ID NO: 114 - DNA, 40 bp, Artificial Sequence; identical to SEQ ID NO: 6
  oligomer

SEQ ID NO: 115 - DNA, 39 bp, Artificial Sequence
  oligomer
nnnnnnnnnnnnnnnnnnnnnnnnnnnnnnnnnnnnnnn

SEQ ID NO: 116 - DNA, 37 bp, Artificial Sequence
  oligomer
nnnnnnnnnnnnnnnnnnnnnnnnnnnnnnnnnnnnn

SEQ ID NO: 117 - DNA, 35 bp, Artificial Sequence
  oligomer
nnnnnnnnnnnnnnnnnnnnnnnnnnnnnnnnnnn

SEQ ID NO: 118 - DNA, 33 bp, Artificial Sequence
  oligomer
nnnnnnnnnnnnnnnnnnnnnnnnnnnnnnnnn

SEQ ID NO: 119 - DNA, 31 bp, Artificial Sequence
  oligomer
nnnnnnnnnnnnnnnnnnnnnnnnnnnnnnn

SEQ ID NO: 120 - DNA, 20 bp, Artificial Sequence
  oligomer
cccccccccccccccccccc

SEQ ID NO: 121 - DNA, 30 bp, Artificial Sequence
  oligomer
cccccccccccccccccccccccccccccc

SEQ ID NO: 122 - DNA, 50 bp, Artificial Sequence
  oligomer
cccccccccccccccccccccccccccccccccccccccccccccccccc

SEQ ID NO: 123 - DNA, 60 bp, Artificial Sequence
  oligomer
cccccccccccccccccccccccccccccccccccccccccccccccccccccccccccc